(19) United States
(12) Patent Application Publication
(10) Pub. No.: US 2026/0363720 A1
(43) Pub. Date: Nov. 19, 2026

(54) Needle Driver with Suture Cutting Function

(71) Applicant: VICARIOUS SURGICAL INC., Waltham, MA (US)

(72) Inventors: Richard Mansfield, Sterling, MA (US); Michael Tinstman, Malden, MA (US); Nandishkumar Desai, North Reading, MA (US)

(21) Appl. No.: 19/144,059

(22) PCT Filed: Dec. 29, 2023
(86) PCT No.: PCT/US2023/086360

§ 371 Date: Jun. 27, 2025

Related U.S. Application Data
(60) Provisional application No. 63/435,889, filed on Dec. 29, 2022.

(51) Int. Cl. A61B 17/285; A61B 17/29

(52) U.S. Cl. CPC A61B 17/285; A61B 2017/2926; A61B 2017/2947

(57) ABSTRACT

Improved end effector mechanisms for a surgical instrument used in minimally invasive surgical instruments as well as instruments for general surgery or as part of robotically controlled end effectors. These end effector mechanisms include multiple grasping elements paired with drive links.

### CROSS-REFERENCE TO RELATED APPLICATIONS

This application claims the benefit of U.S. Provisional Patent Application No. 63/435,889 filed Dec. 29, 2022, the entire contents of which are incorporated by reference herein in their entirety.

### BACKGROUND

Since its inception in the early 1990s, the field of minimally invasive surgery has rapidly grown. While minimally invasive surgery vastly improves patient outcome, this improvement comes at a cost to the surgeon's ability to operate with precision and ease. During conventional laparoscopic procedures, the surgeon inserts a laparoscopic instrument through multiple small incisions in the patient's abdominal wall. The nature of tool insertion through the abdominal wall constrains the motion of the laparoscopic instruments, as the instruments are unable to move side-to-side without injury to the abdominal wall. Conventional laparoscopic instruments are also limited in motion, and are often limited to four axes of motion. These four axes of motion are movement of the instrument in and out of the trocar (axis 1), rotation of the instrument within the trocar (axis 2), and angular movement of the trocar in two planes while maintaining the pivot point of the trocar's entry into the abdominal cavity (axes 3 and 4). For over two decades, the majority of minimally invasive surgery has been performed with only these four degrees of motion. Moreover, prior systems require multiple incisions if the surgery requires addressing multiple different locations within the abdominal cavity.

Existing robotic surgical devices attempted to solve many of these problems. Some existing robotic surgical devices replicate non-robotic laparoscopic surgery with additional degrees of freedom at the end of the instrument. However, even with many costly changes to the surgical procedure, existing robotic surgical devices often do not provide improved patient outcome in the majority of procedures for which they are used. Additionally, existing robotic devices create increased separation between the surgeon and surgical end effectors. This increased separation can cause injuries resulting from the surgeon's misunderstanding of the motion and the force applied by the robotic device. Because the degrees of freedom of many existing robotic devices are unfamiliar to a human operator, surgeons need extensive training on robotic simulators before operating on a patient in order to minimize the likelihood of causing inadvertent injury.

Furthermore, in laparoscopic and robotic surgeries, a variety of tools and graspers are needed to complete a surgical procedure. Initially inserting all of the necessary tools to be employed by the surgical robotic system at once within the patient can result in an increased risk to the patient due to the possible use of excess incision sites and the inherent increased complexity of safely storing and manipulating the tools during the surgical procedure. Thus, conventional surgical procedures often rely on removing and replacing tools throughout the surgery. This removal and replacement process serves to lengthen the time of the surgery, increasing the possibility of complications as tools are fully removed and new tools are inserted within the patient, and increasing the amount of material needed for and hence the potential cost of the surgery.

Specifically, end effector mechanisms, which refer to the portion of a surgical instrument that can contact and manipulate tissue in a patient. End effectors include grasping forceps, which grasp but do not intentionally cut or puncture tissue, as well as cutting tools. These devices replace the surgeon's hands in the traditional open surgery. However, conventional end effectors that are used to perform a cutting action (e.g., cutters), for example cutting a suture, often require the surgeon to instruct the robotic surgical system to switch modes in order to safely engage the cutters. Requiring a specific mode where cutting is enabled adds an extra step to the surgical process. Also, conventional cutters cut when closing the end effector, which raises the risk of accidentally cutting when trying to perform a different task, for example, grasping a needle, tissue, or a suture.

### SUMMARY

Disclosed herein are improved end effector mechanisms and methods for use in minimally invasive surgical instruments, for example, end effectors for robotic surgical systems. The present disclosure teaches end effectors with independently controllable rotatable jaws. The rotatable jaws are controllable to move toward each other to grasp, for example, a needle, and controllable to move away from each other to cut, for example, to cut a suture. The methods taught herein allow control of an end effector for a robotic surgical system. The rotatable jaws of the end effector can be closed to grasp and opened to cut.

In some embodiments taught herein, an end effector for a robotic surgical system includes a first rotatable jaw and second rotatable jaw. The first rotatable jaw is rotatable in a first rotational direction about an axis of rotation to perform a grasping operation and rotatable in a second rotational direction about the axis of rotation to perform a cutting operation. The second rotatable jaw is rotatable in the second rotational direction about the axis of rotation to perform the grasping operation and rotatable in the first rotational direction about the axis of rotation to perform the cutting operation.

### BRIEF DESCRIPTION OF THE DRAWINGS

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 schematically depicts an example surgical robotic system in accordance with some embodiments.

FIG. 2A is an example perspective view of a patient cart including a robotic support system coupled to a robotic subsystem of the surgical robotic system in accordance with some embodiments.

FIG. 2B is an example perspective view of an example operator console of a surgical robotic system of the present disclosure in accordance with some embodiments.

FIG. 3A schematically depicts an example side view of a surgical robotic system performing a surgery within an internal cavity of a subject in accordance with some embodiments.

FIG. 3B schematically depicts an example top view of the surgical robotic system performing the surgery within the internal cavity of the subject of FIG. 3A in accordance with some embodiments.

FIG. 4A is an example perspective view of a single robotic arm subsystem in accordance with some embodiments.

FIG. 4B is an example perspective side view of a single robotic arm of the single robotic arm subsystem of FIG. 4A in accordance with some embodiments.

FIG. 5 is an example perspective front view of a camera assembly and a robotic arm assembly in accordance with some embodiments.

FIG. 6A is an example perspective view of a left hand controller for use in an operator console of a surgical robotic system in accordance with some embodiments.

FIG. 6B is an example perspective view of a right hand controller for use in an operator console of a surgical robotic system in accordance with some embodiments.

FIG. 7 is a perspective view of an example end effector having opposing grasping faces and opposing cutting edges as taught herein.

FIG. 8 is a perspective view of an example end effector illustrating rotational directions of a first rotatable jaw relative to a second rotatable jaw to grasp as taught herein.

FIG. 9 is a perspective view of an example end effector illustrating a fully closed position as taught herein.

FIG. 10 is a perspective view of an example end effector illustrating rotational directions of a first rotatable jaw relative to a second rotatable jaw to cut as taught herein.

FIG. 11 is a perspective view of an example end effector illustrating a cutting position as taught herein.

FIG. 12A is a rear perspective view of a hinge flex body according to one embodiment.

FIG. 12B is a front perspective view of a hinge flex body according to one embodiment.

FIG. 12C is a side perspective view of a hinge flex body according to one embodiment.

FIG. 12D is an additional side perspective view of a hinge flex body according to one embodiment.

FIG. 13A is an isometric view of a hinge non-flex body according to one embodiment.

FIG. 13B is side perspective view of a hinge non-flex body according to one embodiment.

FIG. 13C is a side isometric view of a hinge non-flex body according to one embodiment.

FIG. 13D is a side perspective view of a hinge non-flex body according to one embodiment.

FIG. 14A is a top isometric view of a hinge rotary assembly according to one embodiment.

FIG. 14B is an exploded top profile view of a hinge rotary assembly according to one embodiment.

FIG. 14C is a front perspective view of a rotary pulley body according to one embodiment.

FIG. 15A is an isometric view of a male rotary-hinge body according to one embodiment.

FIG. 15B is an additional isometric view of a male rotary-hinge body according to one embodiment.

FIG. 16A is perspective view illustrating pivot axes formed by mating a hinge flex body and a hinge non-flex body according to one embodiment.

FIG. 16B is a side profile view of a hinge non-flex body illustrating a pivot axis of the end effector.

FIG. 16C is a top profile view of the coupling between a hinge flex body and a hinge non-flex body illustrating a pivot axis.

FIG. 16D illustrates a perspective view of the pivot axes of a robotic arm assembly coupled with an example end effector.

FIG. 17 is a perspective view of an example end effector illustrating rotational directions of a first rotatable jaw relative to a second rotatable jaw to grasp as taught herein.

FIG. 18 is a perspective view of an example end effector illustrating a relative jaw position of a grasping operation as taught herein.

FIG. 19 is a perspective view of an example end effector illustrating rotational directions of a first rotatable jaw relative to a second rotatable jaw to cut as taught herein.

FIG. 20 is a perspective view of an example end effector illustrating a relative jaw position during a cutting operation as taught herein.

FIGS. 21-22 are perspective views of an example end effector illustrating a cutting position as taught herein.

FIG. 23 is a side view of an example end effector illustrating attachment of one rotatable jaw to another as taught herein.

FIGS. 24A, 24B, 24C depict top cross-sectional views of an example cartridge holding an end effector and the insertion of a distal portion of a robotic arm into the cartridge in accordance with some embodiments.

FIG. 25A is a graph depicting the relationship between user inputs and grasping and cutting operations of a needle driver in a continuous control scheme in accordance with some embodiments.

FIG. 25B is a graph depicting the relationship between user inputs and grasping and cutting operations of a needle driver in a discrete control scheme in accordance with some embodiments.

FIG. 25C is a graph depicting the relationship between user inputs and grasping and cutting operations of a needle driver in a discrete zone control scheme in accordance with some embodiments.

FIG. 25D is a graph depicting the relationship between user inputs and grasping and cutting operations of a needle driver in a smooth discrete zone control scheme in accordance with some embodiments.

### DETAILED DESCRIPTION

Taught herein are end effectors for surgical robotic systems that are able to cut or grasp based on a rotational direction of two opposing rotatable jaws. The end effectors include two rotatable jaws that are able to rotate independently of the other or in unison.

To grasp, a first rotatable jaw of the end effector rotates in a first rotational direction and a second rotatable jaw of the end effector rotates in a second rotational direction, opposite to the first rotational direction. To cut, such as a suture, the first rotatable jaw rotates in the second rotational direction and the second rotatable jaw rotates in the first rotational direction opposite to the second rotational direction.

The first and second rotatable jaws each include a grasping face, a cutting face, and a cutting edge. The grasping faces form a first pair of opposing features. The grasping faces oppose each other and function as grasping features to grasp and manipulate tools, tissue, etc. To grasp an object the first pair of opposing features rotate relative to one another in a closing direction.

The cutting face and cutting edge on each of the first and second rotatable jaws form a second pair of opposing features, which together form a cutter or scissors. The cutting edges on the second pair of opposing features are able to cut, for example, a suture as the first pair of features are separated beyond a specified angle. The cutting edges on the second pair of opposing features are rotatable past by each other in such a way that a suture that occupies an area between the opposing second features is cut. The cutting edges on the second pair of opposing features are able to cut when the first and second rotatable jaws are rotated in an opening direction (i.e., grasping faces moving away from each other). To cut an object, the second pair of opposing features rotate relative to one another as the rotatable jaws rotate relative to each other in an opening direction.

During the normal range of motion of the grasping operation for the first pair of opposing features, the second pair of opposing features forming the cutter do not pass by each other, and minimize the risk of accidentally cutting while the first pair or opposing features interact with a tool or tissue. As referred to herein, an aperture angle phi is defined between a grasping face on a first rotatable jaw and a grasping face on a second rotatable jaw. An aperture angle phi of 0° occurs when the grasping face of the first rotatable jaw and the grasping face of the second rotatable jaw are in contact with each other. In some embodiments, a normal range of motion of the grasping operation corresponds to an aperture angle phi of between 0° and 45°. In some embodiments, a normal range of motion for the cutting operation corresponds to an aperture angle phi of between 45° and 75°.

In some embodiments, the two rotatable jaws have an opposing bore which defines a common rotational axis. The two rotatable jaws are connected along the common rotational axis, for example, by an axel.

In some embodiments, the two rotatable jaws are connected by a fastener that extends through the first and second rotatable jaws. Both rotatable jaws rotate about the fastener, which defines an axis of rotation. In some embodiments, one rotatable jaw has a bore through which the fastener extends and one rotatable jaw has a radially shaped channel (race) through which the fastener extends. The rotatable jaw with the radially shaped channel rotates about the fastener via the channel. The radially shaped channel provides a rotational guide for the rotatable jaw.

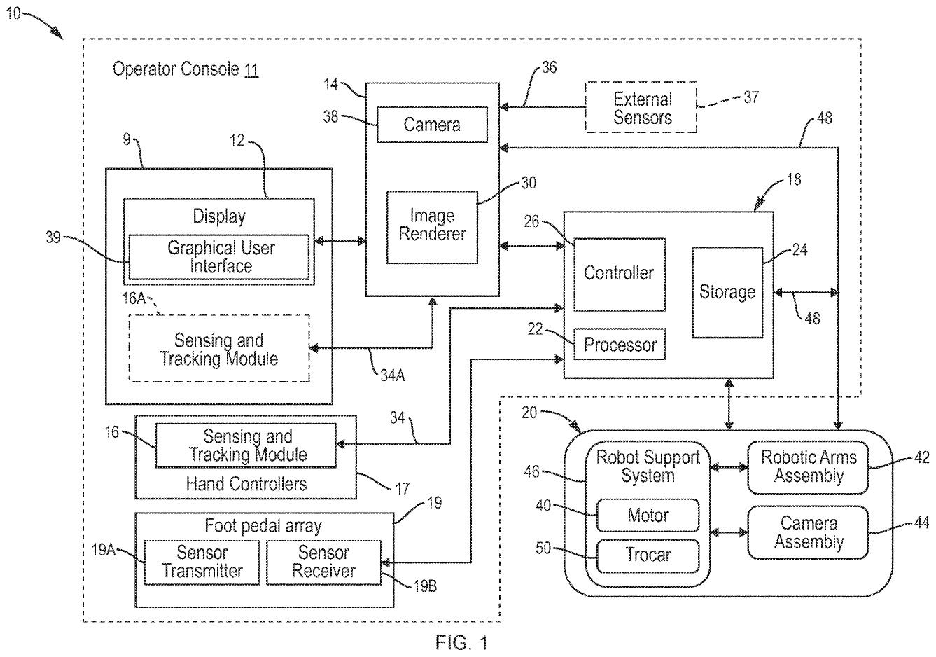

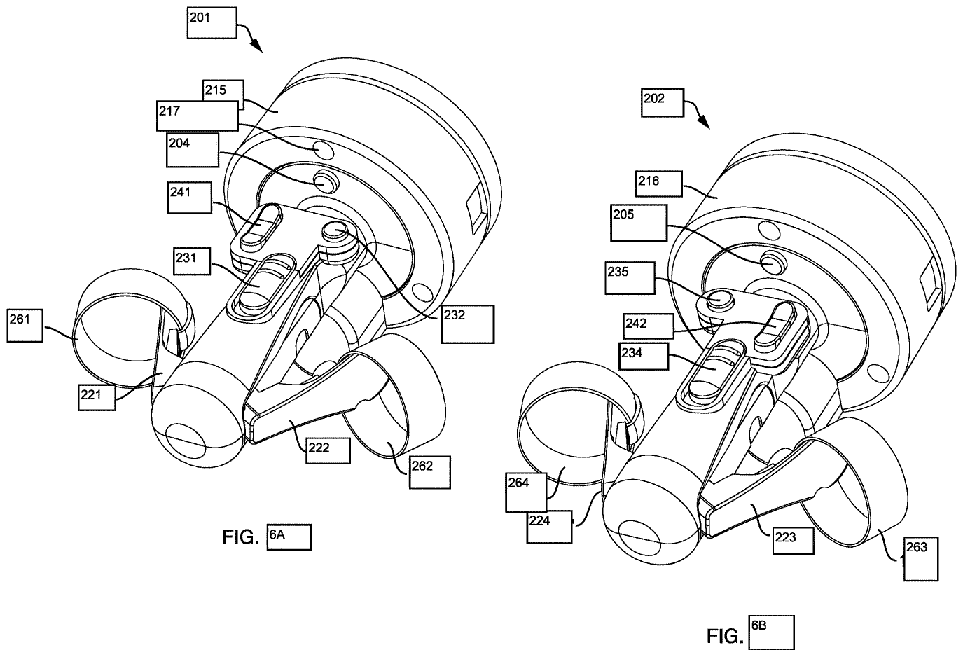

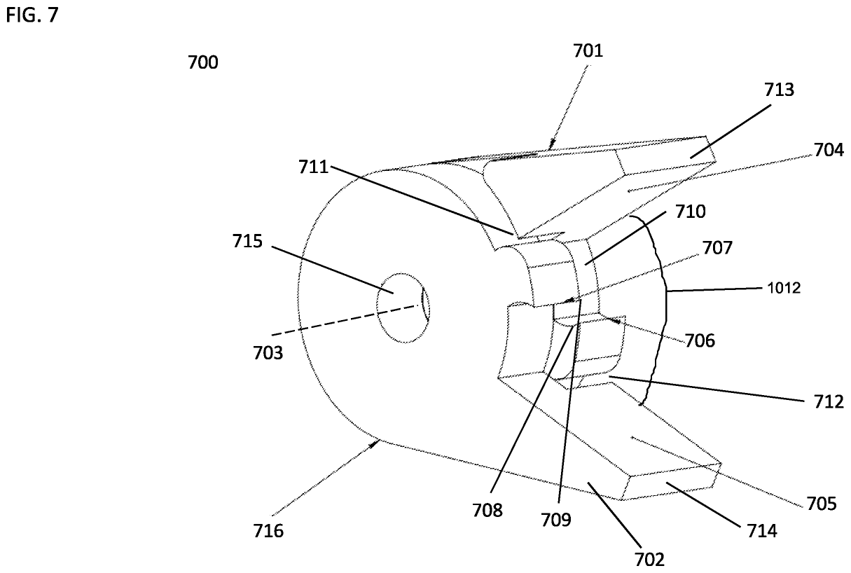

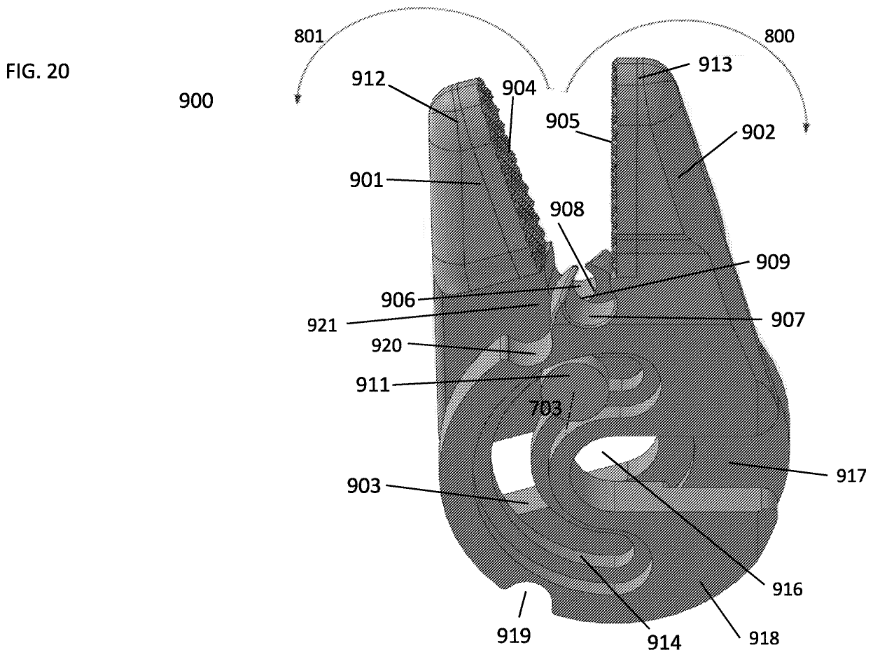

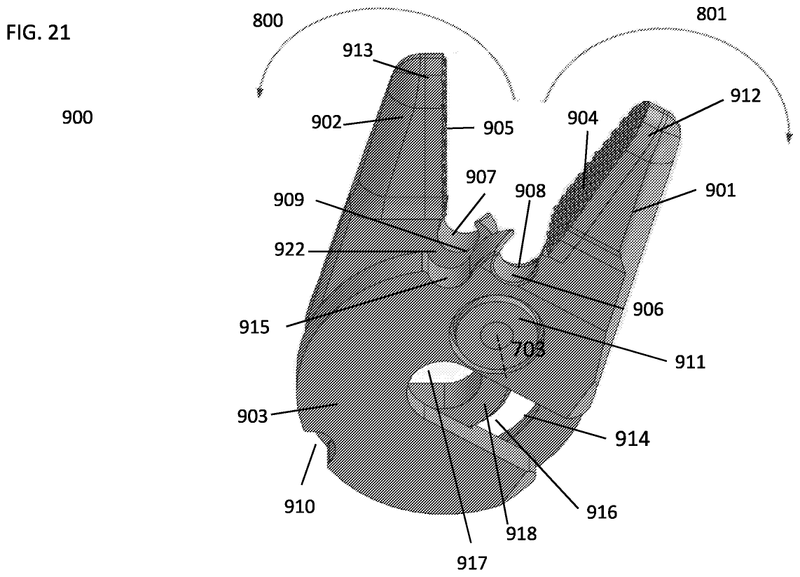

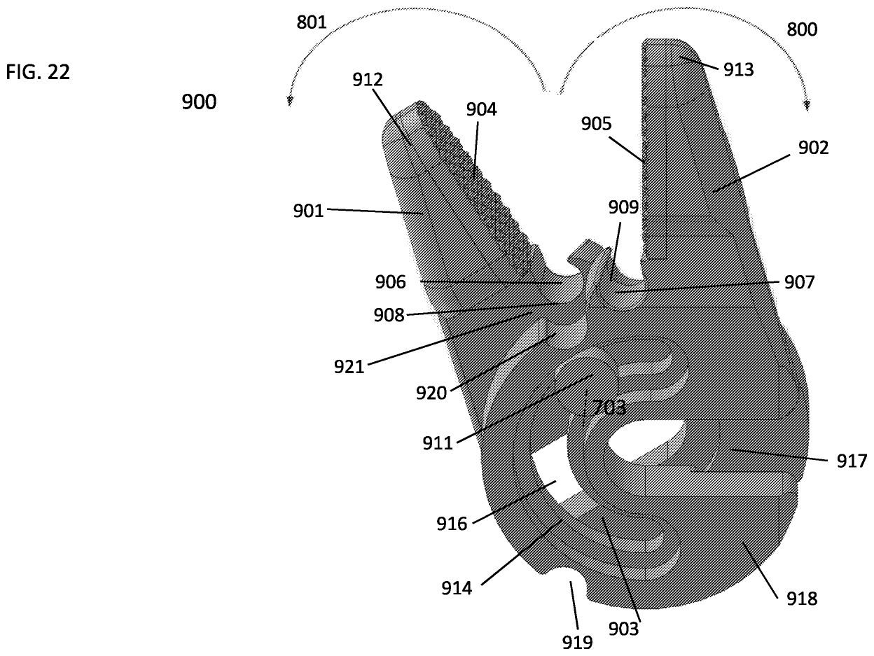

Prior to providing additional specific description of the end effectors as taught herein with respect to FIGS. 7-24C a surgical robotic system in which some embodiments could be employed is described below with respect to FIGS. 1-6B.

While various embodiments of the disclosure have been taught and described herein, it will be clear to those skilled in the art that such embodiments are provided by way of example only. Numerous variations, changes, and substitutions can occur to those skilled in the art without departing from the invention. It can be understood that various alternatives to the embodiments taught herein can be employed.

As used in the specification and claims, the singular form “a,” “an,” and “the” include plural references unless the context clearly dictates otherwise. It will be further understood that the terms “comprises” and/or “comprising,” or “include” and/or “including,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although some example embodiments can be described herein or in documents incorporated by reference as employing a plurality of units to perform example processes, it is understood that example processes can also be performed by one or a plurality of modules. Additionally, it is understood that the term controller/controller can refer to a hardware device that includes a memory and a processor and is specifically programmed to execute the processes described herein in accordance with some embodiments. In some embodiments, the memory is configured to store the modules and the processor is specifically configured to execute said modules to perform one or more processes which are described further below. In some embodiments, multiple different controllers or controllers or multiple different types of controllers or controllers can be employed in performing one or more processes. In some embodiments, different controllers or controllers can be implemented in different portions of a surgical robotic systems.

### Surgical Robotic Systems

Some embodiments can be employed with a surgical robotic system. A system for robotic surgery can include a robotic subsystem. The robotic subsystem includes at least a portion, which can also be referred to herein as a robotic assembly herein, that can be inserted into a patient via a trocar through a single incision point or site. The portion inserted into the patient via a trocar is small enough to be deployed in vivo at the surgical site and is sufficiently maneuverable when inserted to be able to move within the body to perform various surgical procedures at multiple different points or sites. The portion inserted into the body that performs functional tasks can be referred to as a surgical robotic module, a surgical robotic module or a robotic assembly herein. The surgical robotic module can include multiple different submodules or parts that can be inserted into the trocar separately. The surgical robotic module, surgical robotic module, or robotic assembly can include multiple separate robotic arms that are deployable within the patient along different or separate axes. These multiple separate robotic arms can be collectively referred to as a robotic arm assembly herein. Further, a surgical camera assembly can also be deployed along a separate axis. The surgical robotic module, surgical robotic module, or robotic assembly can also include the surgical camera assembly. Thus, the surgical robotic module, or robotic assembly employs multiple different components, such as a pair of robotic arms and a surgical or robotic camera assembly, each of which are deployable along different axes and are separately manipulatable, maneuverable, and movable. The robotic arms and the camera assembly that are disposable along separate and manipulatable axes is referred to herein as the Split Arm (SA) architecture. The SA architecture is designed to simplify and increase efficiency of the insertion of robotic surgical instruments through a single trocar at a single insertion site, while concomitantly assisting with deployment of the surgical instruments into a surgical ready state as well as the subsequent removal of the surgical instruments through the trocar. By way of example, a surgical instrument can be inserted through the trocar to access and perform an operation in vivo in the abdominal cavity of a patient. In some embodiments, various surgical instruments can be used or employed, including but not limited to robotic surgical instruments, as well as other surgical instruments known in the art.

The systems, devices, and methods taught herein can be incorporated into and/or used with a robotic surgical device and associated system taught for example in U.S. Pat. No. 10,285,765 and in PCT patent application Serial No. PCT/US2020/39203, and/or with the camera assembly and system taught in United States Publication No. 2019/0076199, and/or the systems and methods of exchanging surgical tools in an implantable surgical robotic system taught in PCT patent application Serial No. PCT/US2021/058820, where the content and teachings of all of the foregoing patents, patent applications and publications are incorporated herein by reference herein in their entirety. The surgical robotic module that forms part of the present invention can form part of a surgical robotic system that includes a user workstation that includes appropriate sensors and displays, and a robot support system (RSS) for interacting with and supporting the robotic subsystem of the present invention in some embodiments. The robotic subsystem includes a motor and a surgical robotic module that includes one or more robotic arms and one or more camera assemblies in some embodiments. The robotic arms and camera assembly can form part of a single support axis robotic system, can form part of the split arm (SA) architecture robotic system, or can have another arrangement. The robot support system can provide multiple degrees of freedom such that the robotic module can be maneuvered within the patient into a single position or multiple different positions. In one embodiment, the robot support system can be directly mounted to a surgical table or to the floor or ceiling within an operating room. In another embodiment, the mounting is achieved by various fastening means, including but not limited to, clamps, screws, or a combination thereof. In other embodiments, the structure can be free standing. The robot support system can mount a motor assembly that is coupled to the surgical robotic module, which includes the robotic arm assembly and the camera assembly. The motor assembly can include gears, motors, drivetrains, electronics, and the like, for powering the components of the surgical robotic module.

The robotic arm assembly and the camera assembly are capable of multiple degrees of freedom of movement. According to some embodiments, when the robotic arm assembly and the camera assembly are inserted into a patient through the trocar, they are capable of movement in at least the axial, yaw, pitch, and roll directions. The robotic arms of the robotic arm assembly are designed to incorporate and employ a multi-degree of freedom of movement robotic arm with an end effector mounted at a distal end thereof that corresponds to a wrist area or joint of the user. In other embodiments, the working end (e.g., the end effector end) of the robotic arm is designed to incorporate and use or employ other robotic surgical instruments, such as for example the surgical instruments set forth in U.S. Pub. No. 2018/0221102, the entire contents of which are herein incorporated by reference.

Like numerical identifiers are used throughout the figures to refer to the same elements.

FIG. 1 is a schematic illustration of an example surgical robotic system 10 in which aspects of the present disclosure can be employed in accordance with some embodiments of the present disclosure. The surgical robotic system 10 includes an operator console 11 and a robotic subsystem 20 in accordance with some embodiments.

The operator console 11 includes a display 12, an image computing module 14, which can be a three-dimensional (3D) computing module, hand controllers 17 having a sensing and tracking module 16, and a computing module 18. Additionally, the operator console 11 can include a foot pedal array 19 including a plurality of pedals. The image computing module 14 can include a graphical user interface 39. The graphical user interface 39, the controller 26 or the image renderer 30, or both, can render one or more images or one or more graphical user interface elements on the graphical user interface 39. For example, a pillar box associated with a mode of operating the surgical robotic system 10, or any of the various components of the surgical robotic system 10, can be rendered on the graphical user interface 39. Also live video footage captured by a camera assembly 44 can also be rendered by the controller 26 or the image renderer 30 on the graphical user interface 39.

The operator console 11 can include a visualization system 9 that includes a display 12 which can be any selected type of display for displaying information, images or video generated by the image computing module 14, the computing module 18, and/or the robotic subsystem 20. The display 12 can include or form part of, for example, a head-mounted display (HMD), an augmented reality (AR) display (e.g., an AR display, or AR glasses in combination with a screen or display), a screen or a display, a two-dimensional (2D) screen or display, a three-dimensional (3D) screen or display, and the like. The display 12 can also include an optional sensing and tracking module 16A. In some embodiments, the display 12 can include an image display for outputting an image from a camera assembly 44 of the robotic subsystem 20.

The hand controllers 17 are configured to sense a movement of the operator's hands and/or arms to manipulate the surgical robotic system 10. The hand controllers 17 can include the sensing and tracking module 16, circuity, and/or other hardware. The sensing and tracking module 16 can include one or more sensors or detectors that sense movements of the operator's hands. In some embodiments, the one or more sensors or detectors that sense movements of the operator's hands are disposed in the hand controllers 17 that are grasped by or engaged by hands of the operator. In some embodiments, the one or more sensors or detectors that sense movements of the operator's hands are coupled to the hands and/or arms of the operator. For example, the sensors of the sensing and tracking module 16 can be coupled to a region of the hand and/or the arm, such as the fingers, the wrist region, the elbow region, and/or the shoulder region. Additional sensors can also be coupled to a head and/or neck region of the operator in some embodiments. In some embodiments, the sensing and tracking module 16 can be external and coupled to the hand controllers 17 via electricity components and/or mounting hardware. In some embodiments, the optional sensor and tracking module 16A can sense and track movement of one or more of an operator's head, of at least a portion of an operator's head, an operator's eyes or an operator's neck based, at least in part, on imaging of the operator in addition to or instead of by a sensor or sensors attached to the operator's body.

In some embodiments, the sensing and tracking module 16 can employ sensors coupled to the torso of the operator or any other body part. In some embodiments, the sensing and tracking module 16 can employ in addition to the sensors an Inertial Momentum Unit (IMU) having for example an accelerometer, gyroscope, magnetometer, and a motion processor. The addition of a magnetometer allows for reduction in sensor drift about a vertical axis. In some embodiments, the sensing and tracking module 16 also include sensors placed in surgical material such as gloves, surgical scrubs, or a surgical gown. The sensors can be reusable or disposable. In some embodiments, sensors can be disposed external of the operator, such as at fixed locations in a room, such as an operating room. The external sensors 37 can generate external data 36 that can be processed by the computing module 18 and hence employed by the surgical robotic system 10.

The sensors generate position and/or orientation data indicative of the position and/or orientation of the operator's hands and/or arms. The sensing and tracking modules 16 and/or 16A can be utilized to control movement (e.g., changing a position and/or an orientation) of the camera assembly 44 and robotic arm assembly 42 of the robotic subsystem 20. The tracking and position data 34 generated by the sensing and tracking module 16 can be conveyed to the computing module 18 for processing by at least one processor 22.

The computing module 18 can determine or calculate, from the tracking and position data 34 and 34A, the position and/or orientation of the operator's hands or arms, and in some embodiments of the operator's head as well, and convey the tracking and position data 34 and 34A to the robotic subsystem 20. The tracking and position data 34, 34A can be processed by the processor 22 and can be stored for example in the storage 24. The tracking and position data 34 and 34A can also be used by the controller 26, which in response can generate control signals for controlling movement of the robotic arm assembly 42 and/or the camera assembly 44. For example, the controller 26 can change a position and/or an orientation of at least a portion of the camera assembly 44, of at least a portion of the robotic arm assembly 42, or both. In some embodiments, the controller 26 can also adjust the pan and tilt of the camera assembly 44 to follow the movement of the operator's head.

The robotic subsystem 20 can include a robot support system (RSS) 46 having a motor 40 and a trocar 50 or trocar mount, the robotic arm assembly 42, and the camera assembly 44. The robotic arm assembly 42 and the camera assembly 44 can form part of a single support axis robot system, such as that taught and described in U.S. Pat. No. 10,285,765, or can form part of a split arm (SA) architecture robot system, such as that taught and described in PCT Patent Application No. PCT/US2020/039203, both of which are incorporated herein by reference in their entirety.

The robotic subsystem 20 can employ multiple different robotic arms that are deployable along different or separate axes. In some embodiments, the camera assembly 44, which can employ multiple different camera elements, can also be deployed along a common separate axis. Thus, the surgical robotic system 10 can employ multiple different components, such as a pair of separate robotic arms and the camera assembly 44, which are deployable along different axes. In some embodiments, the robotic arm assembly 42 and the camera assembly 44 are separately manipulatable, maneuverable, and movable. The robotic subsystem 20, which includes the robotic arm assembly 42 and the camera assembly 44, is disposable along separate manipulatable axes, and is referred to herein as an SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through the trocar 50 as further described below.

The RSS 46 can include the motor 40 and the trocar 50 or a trocar mount. The RSS 46 can further include a support member that supports the motor 40 coupled to a distal end thereof. The motor 40 in turn can be coupled to the camera assembly 44 and to each of the robotic arm assembly 42. The support member can be configured and controlled to move linearly, or in any other selected direction or orientation, one or more components of the robotic subsystem 20. In some embodiments, the RSS 46 can be free standing. In some embodiments, the RSS 46 can include the motor 40 that is coupled to the robotic subsystem 20 at one end and to an adjustable support member or element at an opposed end.

The motor 40 can receive the control signals generated by the controller 26. The motor 40 can include gears, one or more motors, drivetrains, electronics, and the like, for powering and driving the robotic arm assembly 42 and the cameras assembly 44 separately or together. The motor 40 can also provide mechanical power, electrical power, mechanical communication, and electrical communication to the robotic arm assembly 42, the camera assembly 44, and/or other components of the RSS 46 and robotic subsystem 20. The motor 40 can be controlled by the computing module 18. The motor 40 can thus generate signals for controlling one or more motors that in turn can control and drive the robotic arm assembly 42, including for example the position and orientation of each robot joint of each robotic arm, as well as the camera assembly 44. The motor 40 can further provide for a translational or linear degree of freedom that is first utilized to insert and remove each component of the robotic subsystem 20 through the trocar 50. The motor 40 can also be employed to adjust the inserted depth of each robotic arm of the robotic arm assembly 42 when inserted into the patient 100 through the trocar 50.

The trocar 50 is a medical device that can be made up of an awl (which can be a metal or plastic sharpened or non-bladed tip), a cannula (essentially a hollow tube), and a seal in some embodiments. The trocar 50 can be used to place at least a portion of the robotic subsystem 20 in an interior cavity of a subject (e.g., a patient) and can withdraw gas and/or fluid from a body cavity. The robotic subsystem 20 can be inserted through the trocar 50 to access and perform an operation in vivo in a body cavity of a patient. In some embodiments, the robotic subsystem 20 can be supported, at least in part, by the trocar 50 or a trocar mount with multiple degrees of freedom such that the robotic arm assembly 42 and the camera assembly 44 can be maneuvered within the patient into a single position or multiple different positions. In some embodiments, the robotic arm assembly 42 and camera assembly 44 can be moved with respect to the trocar 50 or a trocar mount with multiple different degrees of freedom such that the robotic arm assembly 42 and the camera assembly 44 can be maneuvered within the patient into a single position or multiple different positions.

In some embodiments, the RSS 46 can further include an optional controller for processing input data from one or more of the system components (e.g., the display 12, the sensing and tracking module 16, the robotic arm assembly 42, the camera assembly 44, and the like), and for generating control signals in response thereto. The motor 40 can also include a storage element for storing data in some embodiments.

The robotic arm assembly 42 can be controlled to follow the scaled-down movement or motion of the operator's arms and/or hands as sensed by the associated sensors in some embodiments and in some modes of operation. The robotic arm assembly 42 include a first robotic arm including a first end effector 700, 900 at distal end of the first robotic arm, and a second robotic arm including a second end effector 700, 900 disposed at a distal end of the second robotic arm. In some embodiments, the robotic arm assembly 42 can have portions or regions that can be associated with movements associated with the shoulder, elbow, and wrist joints as well as the fingers of the operator. For example, the robotic elbow joint can follow the position and orientation of the human elbow, and the robotic wrist joint can follow the position and orientation of the human wrist. The robotic arm assembly 42 can also have associated therewith end regions that can terminate in end effectors 700, 900 that follow the movement of one or more fingers of the operator in some embodiments, such as for example the index finger as the user pinches together the index finger and thumb. In some embodiments, the robotic arm assembly 42 can follow movement of the arms of the operator in some modes of control while a virtual chest of the robotic assembly can remain stationary (e.g., in an instrument control mode). In some embodiments, the position and orientation of the torso of the operator are subtracted from the position and orientation of the operator's arms and/or hands. This subtraction allows the operator to move his or her torso without the robotic arms moving. Further disclosure control of movement of individual arms of a robotic assembly is provided in International Patent Application Publications WO 2022/094000 A1 and WO 2021/231402 A1, each of which is incorporated by reference herein in its entirety.

The camera assembly 44 is configured to provide the operator with image data 48, such as for example a live video feed of an operation or surgical site, as well as enable the operator to actuate and control the cameras forming part of the camera assembly 44. In some embodiments, the camera assembly 44 can include one or more cameras (e.g., a pair of cameras), the optical axes of which are axially spaced apart by a selected distance, known as the inter-camera distance, to provide a stereoscopic view or image of the surgical site. In some embodiments, the operator can control the movement of the cameras via movement of the hands via sensors coupled to the hands of the operator or via hand controllers 17 grasped or held by hands of the operator, thus enabling the operator to obtain a desired view of an operation site in an intuitive and natural manner. In some embodiments, the operator can additionally control the movement of the camera via movement of the operator's head. The camera assembly 44 is movable in multiple directions, including for example in yaw, pitch and roll directions relative to a direction of view. In some embodiments, the components of the stereoscopic cameras can be configured to provide a user experience that feels natural and comfortable. In some embodiments, the interaxial distance between the cameras can be modified to adjust the depth of the operation site perceived by the operator.

The image or video data 48 generated by the camera assembly 44 can be displayed on the display 12. In embodiments in which the display 12 includes an HMD, the display can include the built-in sensing and tracking module 16A that obtains raw orientation data for the yaw, pitch and roll directions of the HMD as well as positional data in Cartesian space (x, y, z) of the HMD. In some embodiments, positional and orientation data regarding an operator's head can be provided via a separate head-tracking module. In some embodiments, the sensing and tracking module 16A can be used to provide supplementary position and orientation tracking data of the display in lieu of or in addition to the built-in tracking system of the HMD. In some embodiments, no head tracking of the operator is used or employed. In some embodiments, images of the operator can be used by the sensing and tracking module 16A for tracking at least a portion of the operator's head.

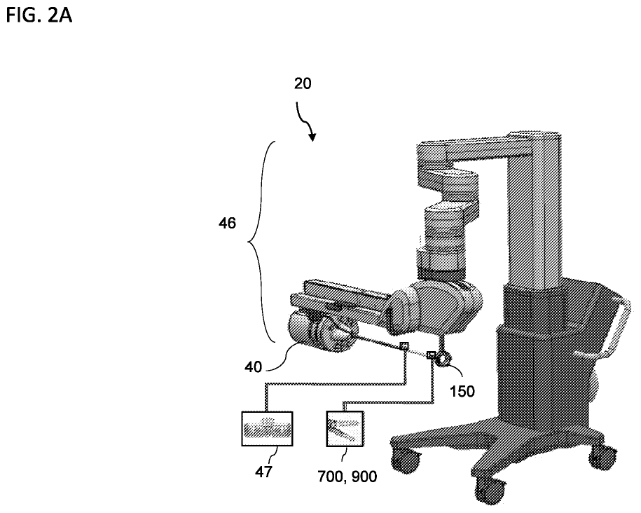

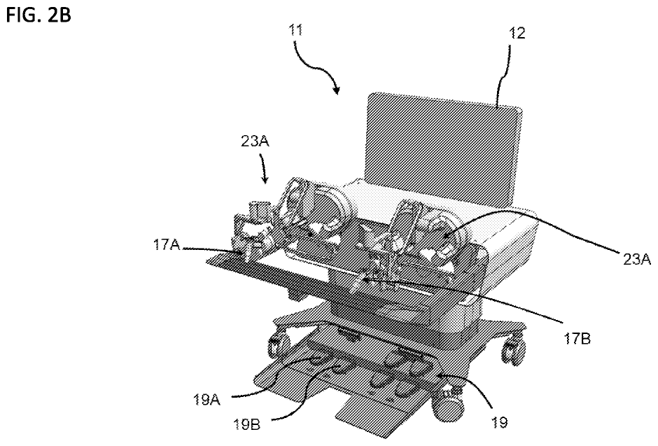

FIG. 2A depicts an example robotic assembly 20, which is also referred to herein as a robotic subsystem, of a surgical robotic system 10 incorporated into or mounted onto a mobile patient cart in accordance with some embodiments. In some embodiments, the robotic subsystem 20 includes the RSS 46, which, in turn includes the motor 40, the robotic arm assembly 42 having end effectors 700, 900, the camera assembly 44 having one or more cameras 47, and can also include the trocar 50 or a trocar mount.

FIG. 2B depicts an example of an operator console 11 of the surgical robotic system 10 of the present disclosure in accordance with some embodiments. The operator console 11 includes a display 12, hand controllers 17, and also includes one or more additional controllers, such as a foot pedal array 19 for control of the robotic arm assembly 42, for control of the camera assembly 44, and for control of other aspects of the system.

FIG. 2B also depicts the left hand controller subsystem 23A and the right hand controller subsystem 23B of the operator console. The left hand controller subsystem 23A includes and supports the left hand controller 17A and the right hand controller subsystem 23B includes and supports the right hand controller 17B. In some embodiments, the left hand controller subsystem 23A can releasably connect to or engage the left hand controller 17A, and right hand controller subsystem 23B can releasably connect to or engage the right hand controller 17A. In some embodiments, the connections can be both physical and electronic so that the left hand controller subsystem 23A and the right hand controller subsystem 23B can receive signals from the left hand controller 17A and the right hand controller 17B, respectively, including signals that convey inputs received from a user selection on a button or touch input device of the left hand controller 17A or the right hand controller 17B.

Each of the left hand controller subsystem 23A and the right hand controller subsystem 23B can include components that enable a range of motion of the respective left hand controller 17A and right hand controller 17B, so that the left hand controller 17A and right hand controller 17B can be translated or displaced in three dimensions and can additionally move in the roll, pitch, and yaw directions. Additionally, each of the left hand controller subsystem 23A and the right hand controller subsystem 23B can register movement of the respective left hand controller 17A and right hand controller 17B in each of the forgoing directions and can send a signal providing such movement information to the processor 22 (as shown in FIG. 1) of the surgical robotic system 10.

In some embodiments, each of the left hand controller subsystem 23A and the right hand controller subsystem 23B can be configured to receive and connect to or engage different hand controllers (not shown). For example, hand controllers with different configurations of buttons and touch input devices can be provided. Additionally, hand controllers with a different shape can be provided. The hand controllers can be selected for compatibility with a particular surgical robotic system or a particular surgical robotic procedure or selected based upon preference of an operator with respect to the buttons and input devices or with respect to the shape of the hand controller in order to provide greater comfort and ease for the operator.

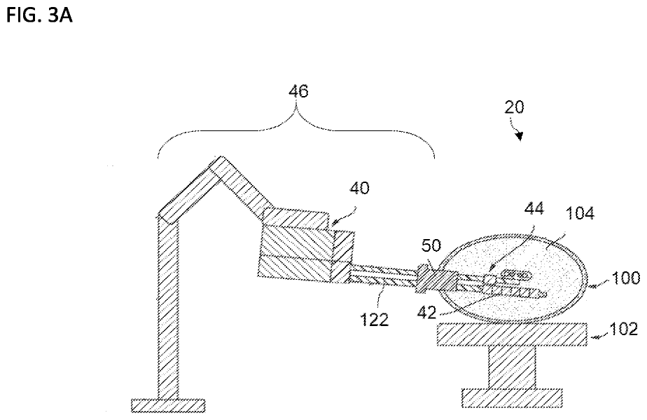

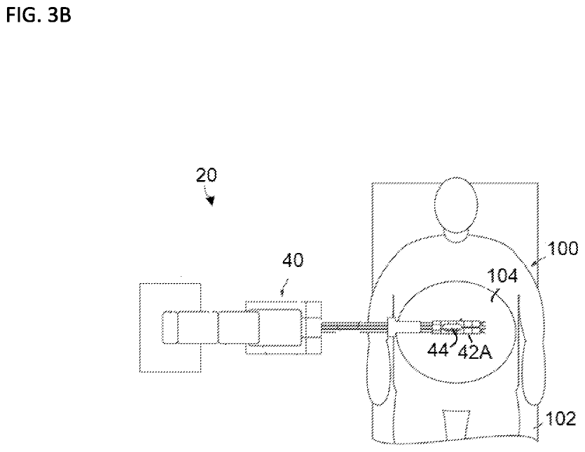

FIG. 3A schematically depicts a side view of the surgical robotic system 10 performing a surgery within an internal cavity 104 of a subject 100 in accordance with some embodiments and for some surgical procedures. FIG. 3B schematically depicts a top view of the surgical robotic system 10 performing the surgery within the internal cavity 104 of the subject 100. The subject 100 (e.g., a patient) is placed on an operation table 102 (e.g., a surgical table 102). In some embodiments, and for some surgical procedures, an incision is made in the patient 100 to gain access to the internal cavity 104. The trocar 50 is then inserted into the patient 100 at a selected location to provide access to the internal cavity 104 or operation site. The RSS 46 can then be maneuvered into position over the patient 100 and the trocar 50. In some embodiments, the RSS 46 includes a trocar mount that attaches to the trocar 50. The camera assembly 44 and the robotic arm assembly 42 can be coupled to the motor 40 and inserted individually and/or sequentially into the patient 100 through the trocar 50 and hence into the internal cavity 104 of the patient 100. Although the camera assembly 44 and the robotic arm assembly 42 can include some portions that remain external to the subject's body in use, references to insertion of the robotic arm assembly 42 and/or the camera assembly 44 into an internal cavity of a patient 100 and disposing the robotic arm assembly 42 and/or the camera assembly 44 in the internal cavity of the patient 100 are referring to the portions of the robotic arm assembly 42 and the camera assembly 44 that are intended to be in the internal cavity of the patient 100 during use. The sequential insertion method has the advantage of supporting smaller trocars and thus smaller incisions can be made in the patient 100, thus reducing the trauma experienced by the patient 100. In some embodiments, the camera assembly 44 and the robotic arm assembly 42 can be inserted in any order or in a specific order. In some embodiments, the camera assembly 44 can be followed by a first robotic arm 42A of the robotic arm assembly 42 and then followed by a second robotic arm 42B of the robotic arm assembly 42 all of which can be inserted into the trocar 50 and hence into the internal cavity 104. Once inserted into the patient 100, the RSS 46 can move the robotic arm assembly 42 and the camera assembly 44 to an operation site manually or automatically controlled by the operator console 11.

Further disclosure regarding control of movement of individual arms of a robotic arm assembly is provided in International Patent Application Publications WO 2022/094000 A1 and WO 2021/231402 A1, each of which is incorporated by reference herein in its entirety.

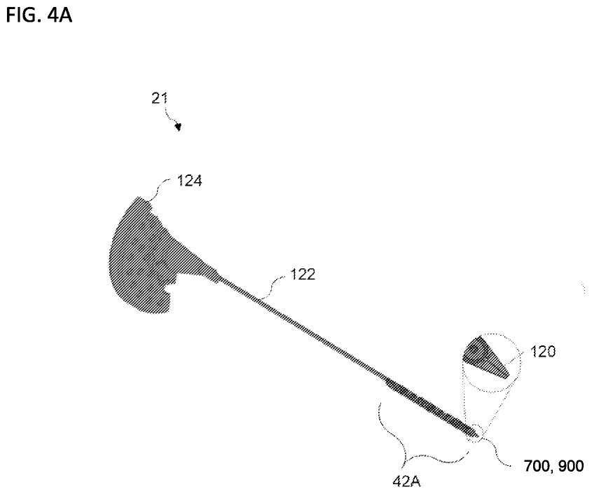

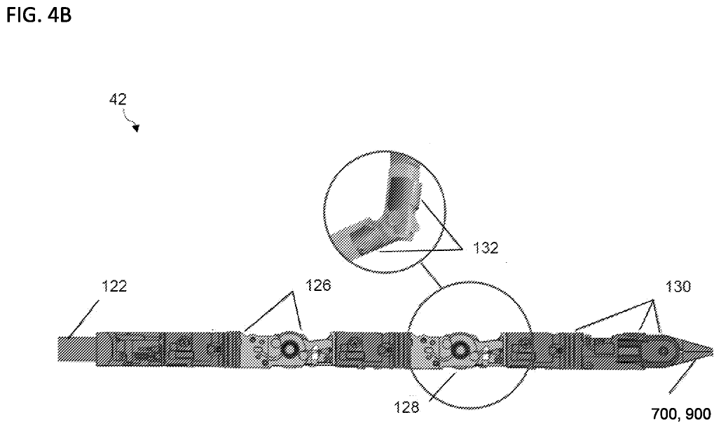

FIG. 4A is a perspective view of a robotic arm subassembly 21 in accordance with some embodiments. The robotic arm subassembly 21 includes a robotic arm 42A, the end effector 700, 900 having an instrument tip 120 (e.g., monopolar scissors, needle driver/holder, bipolar grasper, or any other appropriate tool), a shaft 122 supporting the robotic arm 42A. A distal end of the shaft 122 is coupled to the robotic arm 42A, and a proximal end of the shaft 122 is coupled to a housing 124 of the motor 40 (as shown in FIG. 2A). At least a portion of the shaft 122 can be external to the internal cavity 104 (as shown in FIGS. 3A and 3B). At least a portion of the shaft 122 can be inserted into the internal cavity 104 (as shown in FIGS. 3A and 3B).

FIG. 4B is a side view of the robotic arm assembly 42. The robotic arm assembly 42 includes a shoulder joint 126 forming a virtual shoulder, an elbow joint 128 having position sensors 132 (e.g., capacitive proximity sensors) and forming a virtual elbow, a wrist joint 130 forming a virtual wrist, and the end effector 700, 900 in accordance with some embodiments. The shoulder joint 126, the elbow joint 128, and the wrist joint 130 can include a series of hinge and rotary joints to provide each arm with positionable, seven degrees of freedom, along with one additional grasping degree of freedom for the end effector 700, 900 in some embodiments.

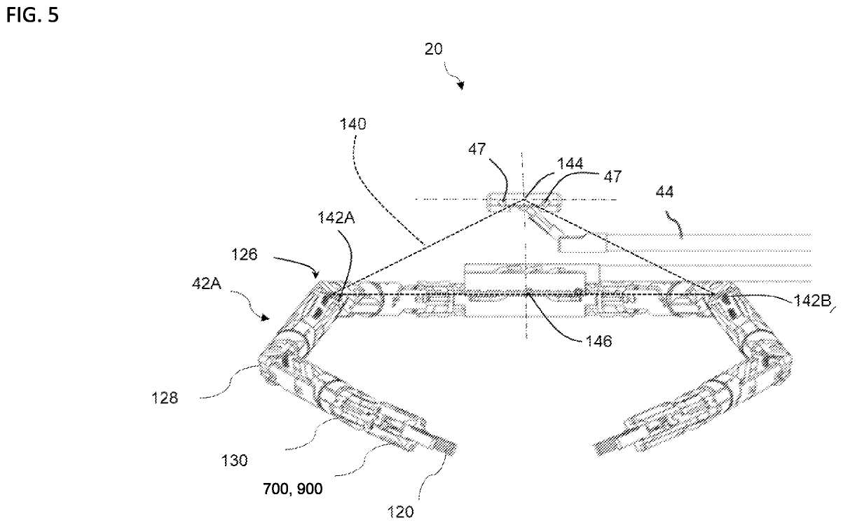

FIG. 5 illustrates a perspective front view of a portion of the robotic assembly 20, which is also referred to herein as a robotic subsystem, configured for insertion into an internal body cavity of a patient. The robotic assembly 20 includes a robotic arm 42A and a robotic arm 42B. The two robotic arms 42A and 42B can define, or at least partially define, a virtual chest 140 of the robotic assembly 20 in some embodiments. In some embodiments, the virtual chest 140 (depicted as a triangle with dotted lines) can be defined by a chest plane extending between a first pivot point 142A of a most proximal joint of the robotic arm 42A (e.g., a shoulder joint 126), a second pivot point 142B of a most proximal joint of the robotic arm 42B, and a camera imaging center point 144 of the camera(s) 47. A pivot center 146 of the virtual chest 140 lies in the middle of the virtual chest 140.

In some embodiments, sensors in one or both of the robotic arm 42A and the robotic arm 42B can be used by the surgical robotic system 10 to determine a change in location in three-dimensional space of at least a portion of each or both of the robotic arms 42A and 42B. In some embodiments, sensors in one or both of the first robotic arm 42A and second robotic arm 42B can be used by the surgical robotic system 10 to determine a location in three-dimensional space of at least a portion of one robotic arm relative to a location in three-dimensional space of at least a portion of the other robotic arm.

In some embodiments, the camera assembly 44 is configured to obtain images from which the surgical robotic system 10 can determine relative locations in three-dimensional space. For example, the camera assembly 44 can include multiple cameras, at least two of which are laterally displaced from each other relative to an imaging axis, and the system can be configured to determine a distance to features within the internal body cavity. Further disclosure regarding a surgical robotic system including camera assembly and associated system for determining a distance to features can be found in International Patent Application Publication No. WO 2021/159409, entitled “System and Method for Determining Depth Perception In Vivo in a Surgical Robotic System,” and published Aug. 12, 2021, which is incorporated by reference herein in its entirety. Information about the distance to features and information regarding optical properties of the cameras can be used by a system to determine relative locations in three-dimensional space.

FIG. 6A depicts a left hand controller 201 and FIG. 6B depicts a right hand controller 202 in accordance with some embodiments. The left hand controller 201 and the right hand controller 202 each include a mounting assembly 215, 216, respectively The mounting assembly 215, 216 can be used to attach, either directly or indirectly, the respective hand controller 201, 202 to a user console of a surgical robotic system. In some embodiments, the mounting assembly 215 defines holes 217, which can be countersunk holes, configured to receive a screw or bolt to connect the left hand controller 201 to a user console.

In some embodiments, such as that depicted in FIGS. 6A and 6B, the hand controller includes two control levers, three buttons, and one touch input device. As will be explained herein, embodiments can feature other combinations of touch input devices, buttons, and levers, or a subset thereof. The hand controllers 201, 202 may have first paddles 221, 223 and second paddles 222, 224 to couple to finger loops 261, 262, 263, 264, respectively. Each finger loop can be a hook and loop (i.e. Velcro®) type loop. In some embodiments (not illustrated), each finger loop can be a hook type. Deflection or depression of the first paddle 221, 223, and the second paddle 222, 224, is configured to trigger a signal to control a tool or an instrument tip (e.g., opening/closing an aperture of graspers/jaws of an instrument tip) at a distal end of a robotic arm of the surgical robotic system. For example, depressing the first paddle 221, 223 and the second paddle 222, 224 can change an angle of jaws of an end effector 700, 900 at a distal end of the respective robotic arm. In some embodiments, end effectors, tools or instruments are used to pull tissue apart, drive a needle driver, grab an item (e.g., a mesh, suture, needle) or pick up such an item in the body cavity when it is dropped, deliver energy via an electrosurgical unit (ESU) (e.g., to cut or to coagulate).

As a non-limiting example, one hand controller may control a grasper and another hand controller may control a needle driver as taught herein. In some embodiments, the hand controller 201 and the paddles 221, 222 control the grasper and the hand controller 202 and the paddles 223, 224 control the needle driver. In other embodiments, the hand controller 201 and the paddles 221, 222 control the needle driver and the hand controller 202 and the paddles 223, 224 control the grasper. The hand controllers 201, 202 may be preprogrammed to control a specific tool or may be programmed to a tool before a surgical operation. The following non-limiting example describes operation of a grasper with the hand controller 201 and the needle driver as taught herein with the hand controller 202.

A surgeon may manipulate the grasper to hold a needle and manipulate the needle driver to cut a suture. The needle driver may be opened or closed by depressing the paddles 223, 224 of controller 202. For example, depressing the paddles 223, 224 may close the tips of the needle driver, and releasing the paddles 223, 224 may open the tips. In some embodiments, a surgeon may partially depress the paddles 223, 224 to sufficiently close the tips to secure a suture, then release the paddles 223, 224 to cut the suture. In some embodiments, a surgeon may partially release the paddles 223, 224 to sufficiently close the tips to secure a suture, then depress the paddles 223, 224 to cut the suture. In some embodiments, a surgeon may partially depress the paddles 223, 224 to sufficiently close the tips to secure a suture, then further depress the paddles 223, 224 to cut the suture. In some embodiments, a surgeon may partially release the paddles 223, 224 to sufficiently close the tips to secure a suture, then further release the paddles 223, 224 to cut the suture.

In some embodiments, a housing of a hand controller can be contoured. In some embodiments, a housing can be shaped to have a contour to match a contour of at least a portion of a thumb of a user's hand. In some embodiments, the housing, the first paddles 221, 223 and the second paddles 222, 224 can each be shaped to comfortably and ergonomically receive a respective hand of a user. In some embodiments, a housing of the hand controller, a paddle or paddles of a hand controller, buttons of a hand controller and/or one or more touch input devices can have shapes and/or positions on the hand controller for fitting different palm sizes and finger lengths.

Left hand controller 201 also includes a first button 231 and a second button 232. Similarly, right hand controller 202 also includes a first button 234 and a second button 235. As taught herein, each button can provide one or more inputs that can be mapped to a variety of different functions of the surgical robotic device to control the surgical robotic system including a camera assembly and a robotic arm assembly. In an embodiment, input received via the first button 231 of the left hand controller 201 and input received via the first button 234 of the right hand controller 202 can control a clutch feature. For example, by engaging the first button 231, 234 a clutch is activated enabling movement of the respective left hand controller 201 or right hand controller 202, by the operator without causing any movement of a robotic arms assembly (e.g., a first robotic arm, a second robotic arm, and a camera assembly) of the surgical robotic system. When the clutch is activated for a hand controller, movement of the respective right hand controller 202 or left hand controller 201 is not translated to movement of the robotic assembly 42. In some embodiments, an operator engaging a hand controller input (e.g., tapping or pressing a button) activates the clutch and the operator engaging again (e.g., tapping or pressing the button again) turns off the clutch or exits a clutch mode. In some embodiments, an operator engaging a hand controller input (e.g., tapping or pressing a button and holding the button) activates the clutch and the clutch stays active for as long as the input is active and exits the clutch when the when the operator is no longer engaging the hand controller input (e.g., releasing the button). Activating the clutch or entering the clutch mode for a hand controller enables the operator to reposition the respective hand controller (e.g., re-position the left controller 201 within the range of motion of the left hand controller 201 and/or re-position the right hand controller 202 within a range of motion of the right hand controller 202) without causing movement of the robotic arm assembly 42 itself. In some embodiments, first buttons 231, 234 may have a slider button type. Sliding the first button 231, 234 may trigger a signal used to control a clutch function for the corresponding hand controller of the surgical robotic system.

The second button 232 of the left hand controller 201 can provide an input that controls a pivot function of the surgical robotic device. An operator engaging (e.g., pressing and holding) the second button 232 of the left hand controller 201 can engage a pivot function or a pivot mode that reorients the robotic arm assembly 42 chest to center the camera on the midpoint between the instrument tips. The pivot function can be activated with a brief tap or held down to continuously track the instrument tips as they move, in accordance with some embodiments.

The second button 235 of the right hand controller 202 can provide input for entering a menu mode in which a menu is displayed on the display 12 of the surgical robotic system 10 and exiting a menu mode. The operator can activate a menu mode by pressing the second button 235 a first time and disengage the menu function by pressing the second button 235 a second time. The operator can be able to select options within the menu by navigating the menu using the left hand controller 201 and/or the right hand controller 202 when the menu mode is engaged. For example, a first touch input device 242 of the right hand controller 202 can be used to navigate the menu and to select a menu item in some embodiments. While in a menu mode, movement of the robotic in response to movement of the left hand controller 201 or the right hand controller 202 can be suspended.

The left hand controller 201 further includes a first touch input device 241. Similarly, the right hand controller 202 further includes a second touch input device 242. In an embodiment, the touch input device 241, 242 can be a three-way switch button type as shown in FIGS. 6A and 6B. Other touch input devices that can be employed include, but are not limited to, scroll wheels, rocker buttons, joy sticks, pointing sticks, touch pads, track balls, trackpoint nubs, etc.

The touch input device 241, 242 can be able to receive input through several different forms of engagement by the operator. For example, where the touch input device 241, 242 is a scroll wheel, the operator can be able to push or click the first touch input device 241, 242, scroll the first touch input device 241, 242 backward or forward, or both.

In some embodiments, scrolling the first touch input device 241 of the left hand controller 201 forward can activate a zoom in function to magnify a view provided by the camera assembly of the surgical robotic system and displayed to the operator, and scrolling backward with first touch input device 241 can provide a zoom out function to reduce the view provided by the camera assembly 44 of the surgical robotic device and displayed to the operator, or vice versa. In embodiments, the zoom function can be mechanical or digital. In some embodiments, the zoom function can be mechanical in part and digital in part (e.g., a mechanical zoom over one zoom range, and a mechanical zoom plus a digital zoom over another zoom range).

In some embodiments, where the touch input device 241, 242 is a three-way switch button, switching or holding the touch input device 241 in the center may trigger a signal used to engage or disengage a scan mode of the surgical robotic system. Switching the touch input device 241 forward may activate a zoom in function to magnify a view provided by the camera assembly of the surgical robotic system and displayed to the operator, and switching backward with first touch input device 241 may provide a zoom out function to reduce the view provided by the camera assembly of the surgical robotic device and displayed to the operator, or vice versa. Switching the second button 235 upward may trigger a signal used to traverse a menu when the menu is displayed or a menu mode is active. Touch input device 242 for the right hand controller 202 may have a three-way switch button type. Switching the touch input device 242 may trigger a signal used to traverse a menu or highlight a portion of the menu when the menu is displayed or a menu mode is active by pressing the second button 235. Switching forward on touch input device 242 may move up the menu and switching backwards with touch input device 242 may move down the menu, or vice versa. Clicking first touch input device 242 may trigger a signal used to select a highlighted portion or of the menu or feature on the menu when the menu is displayed. In some embodiments, switching the touch input device 242 may trigger a signal used to select right elbow bias when the elbow bias function is activated using the menu.

In some embodiments, clicking or depressing first touch input device 241 can engage a scan mode of the surgical robotic system. When in a scan mode, a movement of at least one of the left hand controller 201 or the right hand controller 202 causes a corresponding change in an orientation of a camera assembly 44 of the robotic arm assembly 42 without changing a position or orientation of either robotic arm of the surgical robotic system. In another embodiment, pressing and holding the first touch input device 241 can activate the scan mode and releasing the first touch input device 241 can end the scan mode of the surgical robotic system. In some embodiments, releasing the scan mode returns the camera to the orientation it was in upon entering scan mode. In some embodiments, a function can be provided for locking the orientation upon exiting the scan mode (e.g., to change the “horizon” line).

In some embodiments, when in a menu mode and a left elbow menu item is selected, the first touch input device 241 of the left hand controller 201 can be used for selection of a direction and degree of left elbow bias. As used herein, elbow bias refers to the extent by which the virtual elbow of the robotic arm is above or below a neutral or default position.

In some embodiments, when in a menu mode, an operator can be able to select options within the menu by navigating the menu using the left hand controller 201 and/or the right hand controller 202. For example, when in the menu mode, the second touch input device 242 (e.g., three-way switch or scroll wheel) of the right hand controller 202 provide a set of inputs for traversing a displayed menu and selecting an item in a displayed menu. For example, by scrolling forward on touch input device 242 the operator can move up the menu and by scrolling backwards with touch input device 242 the user can move down the menu, or vice versa. In an embodiment, by clicking first touch input device 242 the operator can make a selection within a menu.

In some embodiments, the second touch input device 242 of the right hand controller 202 can be used to control right elbow bias when a right elbow bias menu item has been selected.

Functions of various buttons and the touch input device described above with respect to the left hand controller 201 above can instead be assigned to the right hand controller 202, and functions of various buttons and the touch input device described above with respect to the right hand controller 202 can instead be assigned to the left hand controller 201 in some embodiments.

The foot pedal array 19 may include a first foot pedal and second foot pedal for receiving operator input. In some embodiments the first foot pedal engages a camera control mode, also described herein as a view control mode, an image framing control mode, or a camera framing control mode of the surgical robotic system and the second foot pedal engages a travel control mode of the surgical robotic system.

In some embodiment, when the camera control mode is activated e.g., using the foot pedal, movement of the left hand controller 201 and/or the right hand controller 202 by the operator can provide input that is interpreted by the system to control a movement of and an orientation of a camera assembly 44 of the surgical robotic system while keeping positions of instrument tips of robotic arms of the robotic arm assembly 42 constant.

In some embodiments, when the travel control mode is activated e.g., using the foot pedal, the left hand controller 201 and the right hand controller 202 can be used to move the robotic arm assembly 42 of the surgical robotic system in a manner in which distal tips of the robotic arms direct or lead movement of a chest of the robotic arm assembly 42 through an internal body cavity. In the travel control mode, a position and orientation of the camera assembly, of the chest, or of both is automatically adjusted to maintain the view of the camera assembly 44 directed at the tips (e.g., at a point between a tip or tips of a distal end of the first robotic arm 42A and a tip or tips of a distal end of the second robotic arm 42B). This can be described as the camera assembly 44 being pinned to the chest of the robotic arm assembly 44 and automatically following the tips.

In some embodiments, the hand controllers as taught herein may not include engage/disengage function. Instead, an operator may put his/her head close to a display such that his/her head may enter a surgeon vigilance sensor range, the operator can squeeze paddles as taught herein to engage an instrument control mode. The operator may pull his/her head out of the surgeon vigilance sensor range to disengage the instrument control mode. In some embodiments, a pivot mode may be deprecated in the surgical robotic system. For example, functionalities of a pivot mode can be consolidated into a camera mode. The camera mode can have 3 degrees of freedom for controlling. The camera mode can be engaged by hitting the second button 232 on the hand controller 201 and then manipulating the hand controllers 201, 202 with 3 degrees of freedom. In some embodiments, the direction of the hand controller movement can be opposite to the direction of chest movement. In some embodiments, paddles 221-224 and/or finger loops 261-264 of hand controllers 201, 202 can be automatically adjusted to be aligned with instrument tips and/or end effectors at all times. In some embodiments, a scan mode can have 2 degrees of freedom for controlling without a rolling degree of freedom.

Although various example embodiments described herein assign certain functions to certain buttons and to certain touch input devices, one of ordinary skill of the art in view of the present disclosure will appreciate that which functions are ascribed to which buttons and touch input devices may be different in different embodiments. Further, one of ordinary skill of the art in view of the present disclosure will appreciate that additional functions not explicitly described herein may assigned to some buttons and some touch input devices in some embodiments. One of ordinary skill of the art in view of the present disclosure will also appreciate that some embodiments may not assign some of the functions described herein or any of the functions described herein to any of the buttons and/or touch input devices of hand controllers. In some embodiments, one or more functions may be assigned to a foot pedal of a surgical robotic system that includes one or more hand controllers as described herein.

### End Effector Mechanism

Discussed below are end effectors for a robotic surgical system as discussed above. The end effectors include rotatable jaws, for example, grasper jaws or needle driver jaws. Like numerical identifiers are used throughout the figures to refer to the same elements.

FIG. 7 illustrates an end effector 700 as taught herein. The end effector 700 includes a first rotatable jaw 701 opposed to a second rotatable jaw 702 that are pivotally coupled to an axel (not shown) extending through a first bore 715 of the first rotatable jaw 701 and a second bore 715′(not shown) of the second rotatable jaw 702. The central portion of each of the first and second bores 715, 715′ defines a common axis of rotation 703. The end effector 700 is adapted to grasp tissue and other material therebetween or cut tissue or material depending on how the first and second rotatable jaws 701, 702 are rotated relative to each other. The first rotatable jaw 701 includes a first grasping face 704, a first cutting face 706, a first cutting edge 708, and the first bore 715 extending through a body portion 710. The second rotatable jaw 702 includes a second grasping face 705, a second cutting face 707, a second cutting edge 709, and the second bore 715′ extending through a body portion 716. The first rotatable jaw 701 and the second rotatable jaw 702 are aligned to each other relative to the first and second bores 715, 715′ to facilitate coupling to the axel, for example a fulcrum about which each of the rotatable jaws rotate. In some embodiments, either the first rotatable jaw 701, the second rotatable jaw 702, or both, include a textured surface on the grasping face 704, 705. The first and second grasping faces 704, 705 form a first pair of opposing features of the end effector 700. The first cutting face 706 and the first cutting edge 708 together with the second cutting face 707 and the second cutting edge 709 form a second pair of opposing features of the end effector 700.

The first grasping face 704 includes a notched or cutout portion 711 located distally from a tip portion 713. Likewise, the second grasping face 705 includes a notched or cutout portion 712 located distally from a tip portion 714. The notched portions 711, 712 of the first and second grasping faces 704, 705 allow rotational movement of the cutting face 706, 707 on the opposing jaw. For example, the first grasping face 704 includes the notch 711 which allows the second cutting face 707 to rotate without interfering with the first rotatable jaw 701. Further, having the first and second cutting faces 706, 707 and, in turn, the first and second cutting edges 708, 709 set back from the tip portion 713, 714 of each of the first grasping face 704 and the second grasping face 705 keeps the cutting features of the end effector 700 out of the way of a grasping operation. The setback location of the first and second cutting faces 706, 707 and the first and second cutting edges 708, 709 helps avoid accidental or unintentional cutting during a grasping operation.

In some embodiments, the cutting faces 706, 707 are contoured. For example, the cutting faces 706, 707 have a curved or helical shape to facilitate holding of a suture during a cutting operation. In some embodiments, the cutting faces 706, 707 are substantially flat.

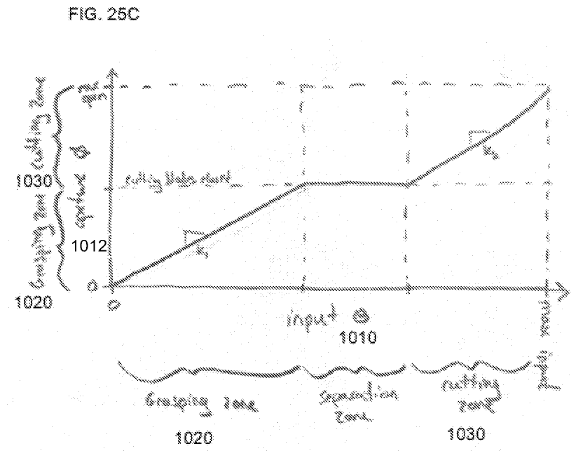

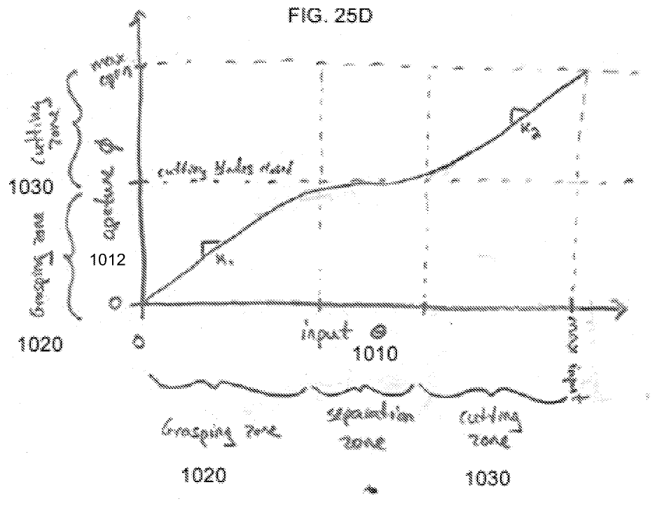

The end effector 700/900 and associated surgical robotic system 10 may be configured to allow a user to control grasping and cutting operations. In some embodiments, for example as depicted in FIG. 25A, a user has continuous control between the grasping and cutting operations. In other words, input from the user 1010 (i.e. manipulation of hand controller 201, 202 at an angle theta) is linearly mapped to an instrument aperture angle 1012 of the end effector 700/900 with a constant scaling factor, k. The user input 1010 defines a grasping zone 1020 and a cutting zone 1030, the boundary between which is defined by the instrument aperture angle 1012 (depicted as “phi” in FIGS. 25A-25D) at which the cutting edges 708, 709, 908, 909 are in contact, e.g. at an instrument aperture angle of forty-five degrees, or anywhere between thirty to fifty-five degrees. In other words, the cutting edges 708, 709, 908, 909 are in contact once the user input 1010 maps to the instrument aperture angle 1012 defining the cutting zone 1030, which in some embodiments may be forty-five degrees.

During operation of the end effector 700/900 in the grasping zone 1020 the opposing features forming the cutter do not pass by each other, and minimize the risk of accidentally cutting while performing the grasping operation. As described in further detail below, the features forming the cutter may be at least partially shielded by the opposing jaw during a grasping operation, for example during operation of the end effector 700/900 in the grasping zone 1020. During operation of the end effector 700/900 in the cutting zone 1030, the opposing features forming the cutter pass by each other and are not shielded.

In some embodiments, for example as depicted in FIG. 25B, the end effector 700/900 and associated surgical robotic system 10 may be configured to operate in a discrete mode wherein the user directly selects between a grasping operation and a cutting operation. The user may choose which mode via an on screen user interface, or with a physical button (i.e., a button 231, 232, 234, 235), or with a foot pedal, or another input mechanism, or with some combination of menus, switches, foot pedals and other input mechanisms. This configuration may enable the full utilization of the input range for each mode, potentially giving the user more fine control. In each mode there can be a discrete scaling factor for each of the grasping mode 1020 and the cutting zone 1030, k1 and k2. K1 and k2 can be the same or can be different depending on the geometry of the input and output, the desired granularity of control, or comfort of the user. When the user changes a mode the system 10 can automatically move the jaws 701, 702, 901, 902 to the appropriate aperture angle 1012 or prompt the user to first control the jaws 701, 702, 901, 902 to the create the appropriate aperture angle 1012.

In some embodiments, for example as depicted in FIG. 25C, the end effector 700/900 and associated surgical robotic system 10 may be configured to allow the user to use both cutting and grasping operations without having to separately choose a mode while also maintaining a separation between cutting and grasping inputs. User inputs may be divided into three zones: grasping 1020, separation 1040 (“dead zone”) and cutting 1030 zones. The separation zone 1040 may be configured to prevent the user from accidentally slipping from the grasping zone 1020 to the cutting zone 1030 and gives the user greater control over the grasping and cutting operations. It requires the user to provide a continuous or accumulated input to go through the separation zone 1040 to change zones. For example, a user may need to provide an input for two or more seconds to initiate movement of the jaws 701, 702, 901, 902 from a grasping position to a cutting position, and vice versa. The features forming the cutter may be at least partially shielded by the opposing jaw during operation of the end effector 700/900 in the separation zone 1040. In each of the grasping 1020 and cutting 1030 zones there can be a discrete scaling factor between the input and output, k1 and k2. K1 and k2 can be the same or can be different depending on the geometry of the input and output, the desired granularity of control, or comfort of the user. As depicted in FIG. 25D, the transition between zones may be smoothed out to provide for a more fluid experience for the user. For example, the time to transition between movement of the jaws 701, 702, 901, 902 from a grasping position to a cutting position may be reduced as compared to FIG. 25C.

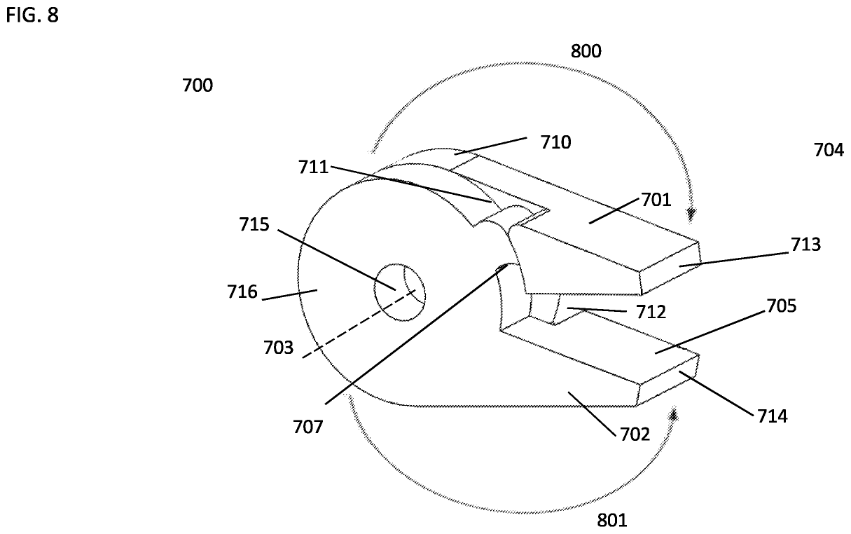

FIG. 8 is another perspective of the end effector 700. In FIG. 8, the grasping operation is illustrated. The first rotatable jaw 701 is rotating in the first rotational direction 800, for example, a clockwise direction. The second rotatable jaw 702 is rotating in the second rotational direction 801, for example, a counterclockwise direction. In other words, the first grasping face 704 of the first rotatable jaw 701 and the second grasping face 705 of the second rotatable jaw 702 are rotating toward each other in a closing direction to grasp or clamp an object between the first grasping face 704 and the second grasping face 705. In some embodiments, the first grasping face 704 of the first rotatable jaw 701 is able to rotate toward the second grasping face 705 while the second rotatable jaw 702 is stationary in order to grasp an object. Likewise, in some embodiments, the second grasping face 705 of the second rotatable jaw 702 is able to rotate toward the first grasping face 704 while the first rotatable jaw 701 is stationary in order to grasp an object.

During the grasping operation, the cutting faces 706, 707 do not pass by each other. In addition, during the grasping operation, the second cutting face 707 rotates in the second rotational direction 801 while the first cutting face 706 rotates in the first rotational direction 800, thereby rotating away from each other. The first cutting face 706 is disposed below a portion of the second grasping face 705. Likewise, the second cutting face 707 is disposed below a portion of the first grasping face 704. As such, during a grasping operation, the first grasping face 704 rotates over and past the second cutting face 707 and the second grasping face 705 rotates over and past the first cutting face 706. The positioning and location of the cutting faces 706, 707 relative to the grasping faces 704, 705 allows the cutting edges 708, 709 to be displaced from the grasping faces 704, 705 to avoid accidental cutting during a grasping operation.

In some embodiments, during the grasping operation the instrument aperture angle phi 1012 between the first grasping face 704/904 and the second grasping face 705/905 may be between zero to thirty degrees. In some embodiments, during the grasping operation the aperture angle phi 1012 between the first grasping face 704/904 and the second grasping face 705/905 may be between zero to forty degrees. In some embodiments, during the grasping operation the aperture angle phi 1012 between the first grasping face 704/904 and the second grasping face 705/905 may be between zero to forty-five degrees. In some embodiments, during the grasping operation the aperture angle phi 1012 between the first grasping face 704/904 and the second grasping face 705/905 may be between zero to fifty degrees. In some embodiments, during the grasping operation the aperture angle phi 1012 between the first grasping face 704/904 and the second grasping face 705/905 may be between zero to fifty-five degrees.

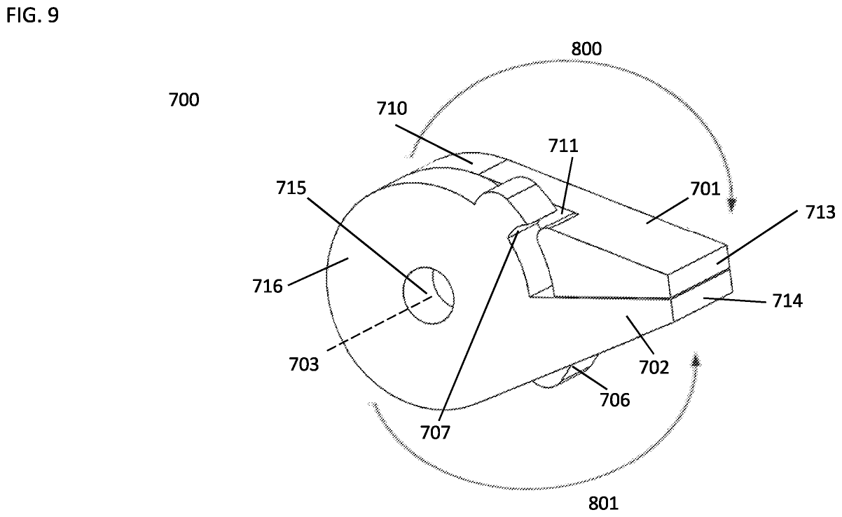

FIG. 9 illustrates the end effector 700 in a fully closed state. As illustrated in FIG. 9, the cutting faces 706, 707 are located on opposite sides of the first and the second grasping faces 704, 705. As such the cutting faces 706, 707 are spaced apart and in some positions partially shielded by the opposing jaw because of the notched or cutout portion 711, 712 during the grasping operation. The first and the second grasping faces 704, 705 include the notched areas 711, 712 allowing first and the second grasping faces 704, 705 to rotate past the cutting faces 706, 707 during the grasping operation. The first and second rotatable jaws 701, 702 are able to rotate in a manner to perform a grasping operation and simultaneously rotate the cutting faces 706, 707 out of the way during the grasping operation. The first and the second rotatable jaws 701, 702 close toward each other to receive tissue and other material therebetween.

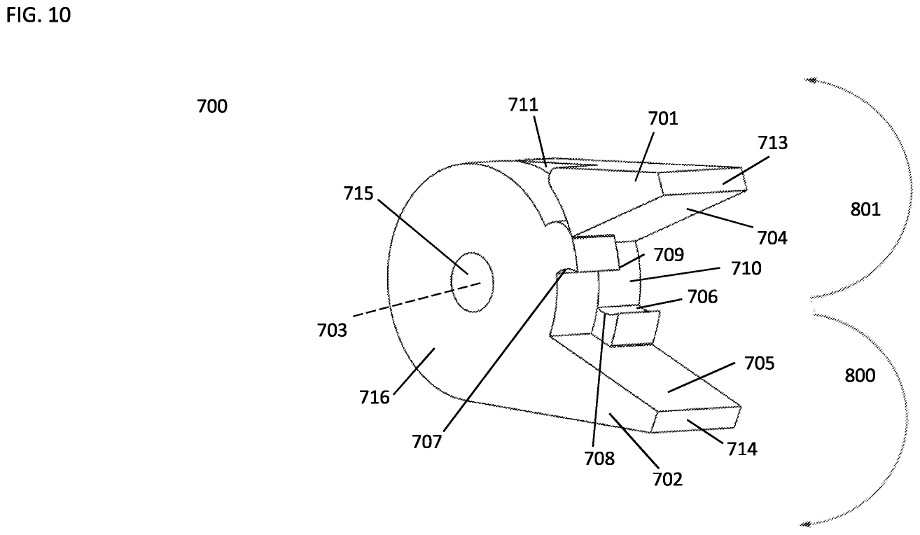

FIG. 10 illustrates the first rotatable jaw 701 rotating in the second rotational direction 801 and the second rotatable jaw 702 rotating in the first rotational direction 800 to perform the cutting operation of the end effector 700. As in the grasping operation, the first and the second rotatable jaws 701, 702 rotate relative to each other. As mentioned above, the first rotatable jaw 701 and the second rotatable jaw 702 are able to rotate independently of each other or in unison. In some embodiments, during the cutting operation, the first cutting face 706 rotates in the second rotational direction 801 and the second cutting face 707 rotates in the first rotational direction 800. In some embodiments, during the cutting operation, the first cutting face 706 rotates in the second rotational direction 801 and the second cutting face 707 does not rotate. In some embodiments, during the cutting operation, the first cutting face 706 does not rotate and the second cutting face 707 rotates in the first rotational direction 800. Whereas during the grasping operation the cutting faces 706, 707 are rotated away from each other, during the cutting operation the cutting faces 706, 707 are rotated toward each other and eventually past each other in order to cut a suture or tissue.

In some embodiments, when the output aperture angle 1012 between the first grasping face 704/904 and the second grasping face 705/905 is between thirty to ninety degrees, the suture or tissue to be cut can be situated between the cutting faces 706, 707, 906, 907. In some embodiments, when the output aperture angle 1012 between the first grasping face 704/904 and the second grasping face 705/905 is between forty-five to seventy-five degrees, the suture or tissue to be cut can be situated between the cutting faces 706, 707, 906, 907. In some embodiments, the cutting faces 706, 707, 906, 907 move past each other to cut a suture or tissue when the output aperture angle 1012 between the first grasping face 704/905 and the second grasping face 705/905 is more than thirty degrees. In some embodiments, the cutting faces 706, 707, 906, 907 move past each other to cut a suture or tissue when the output aperture angle 1012 between the first grasping face 704/904 and the second grasping face 705/905 is more than forty-five degrees. In some embodiments, the cutting faces 706, 707, 906, 907 move past each other to cut a suture or tissue when the output aperture angle 1012 between the first grasping face 704/904 and the second grasping face 705/905 is more than fifty-five degrees. In some embodiments, the first and second rotatable jaws 701, 702, 901, 902 are able to open about 90 degrees relative to each other. This is advantageous because it decreases the chances of the surgeon accidentally cutting a suture or tissue.

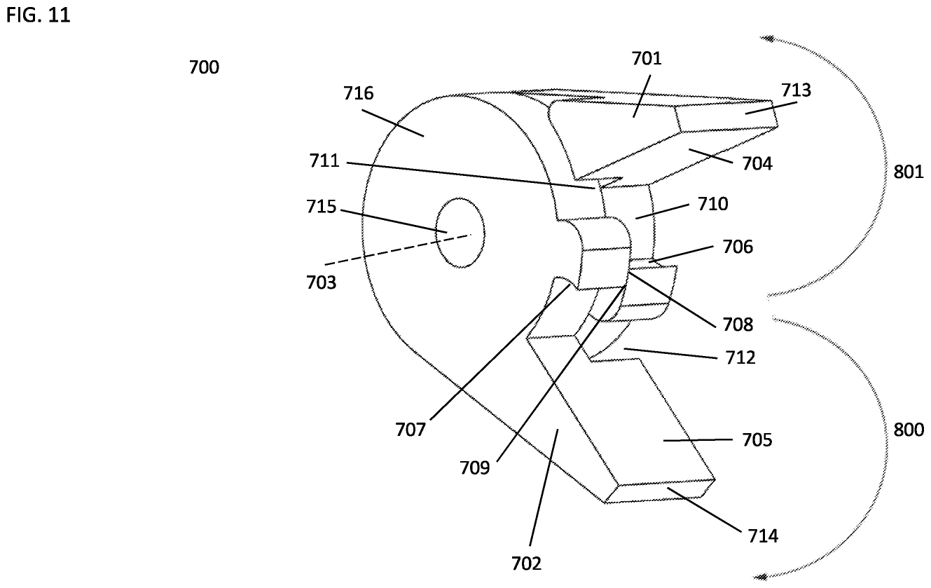

FIG. 11 illustrates rotation of the cutting faces 706, 707 past each other to cut a suture or tissue. As the first and the second rotatable jaws 701, 702 rotate in the second rotational direction 801 and the first rotational direction 800 respectively, the cutting faces 706, 707 rotate past each other to cut, for example, a suture. As the cutting faces 706, 707 rotate past each other, the object resting between the cutting faces 706, 707 (such as a suture) is cut by the cutting edge 708, 709.

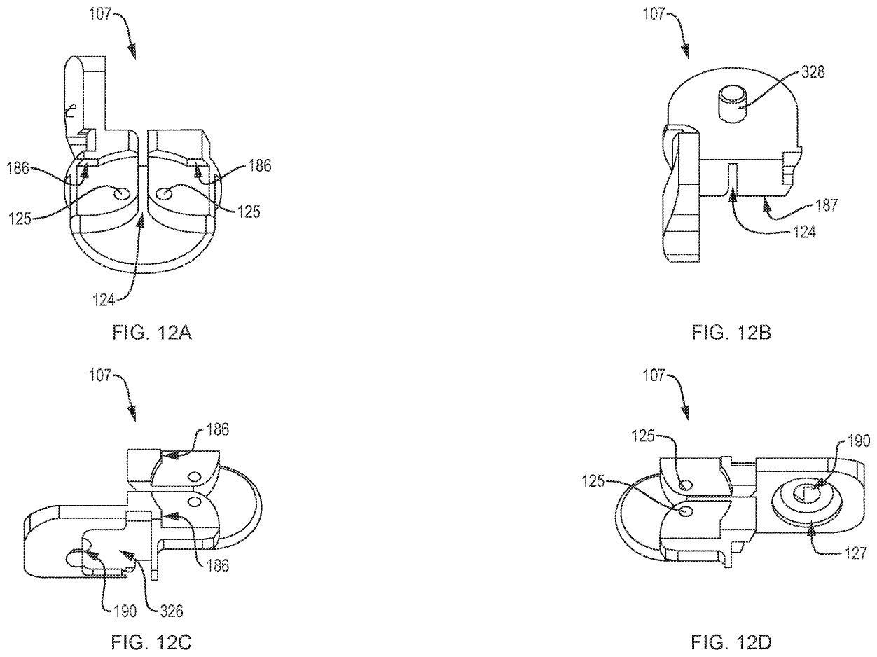

With respect to the end effector described in FIGS. 7-11, below is a description of an embodiment of a suitable attachment mechanism to a distal end of a robotic arm. The attachment mechanism allows the end effector 700 to connect and disconnect from the distal end of the robotic arm. The end effector 700 can include other features and operations not shown in FIGS. 7-11, but are discussed below in relation to FIGS. 12A-16D. Those skilled in the art will appreciate that the jaws of the end effector 700 described above may include other features to facilitate coupling to a distal end of a robotic arm as discussed below.

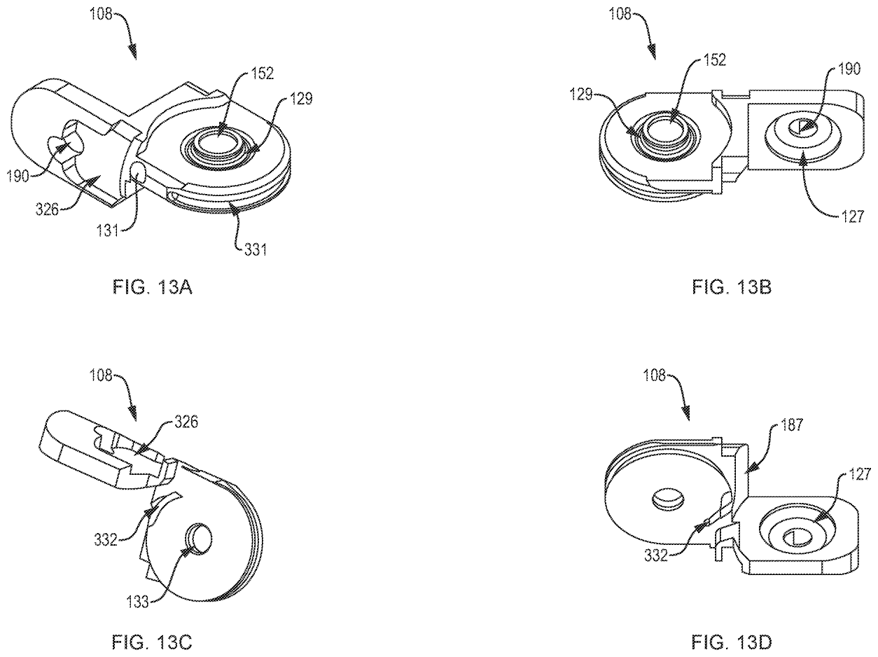

In some embodiments, a hinge flex body 107 and a hinge non-flex body 108 function as a housing for the first rotatable jaw 701, and the second rotatable jaw 702. FIGS. 12A-12D show multiple views of an illustrative embodiment of the hinge flex body 107. FIGS. 13A-13D show multiple views of an illustrative embodiment of the hinge non-flex body 108.

In some embodiments, the first rotatable jaw 701 includes a bearing race. As illustrated in FIGS. 12A-12D in some embodiments, the hinge flex body 107 includes a proximal end and a distal end, and the ends include inner and outer surfaces. In some embodiments, a hinge bearing race 127 is situated on an inner surface of the distal end of the hinge flex body 107. The hinge bearing race 127 is configured to mate and couple with the bearing race of the first rotatable jaw 701. In addition to functioning as a housing for a plurality of ball bearings, the mating between the bearing race of the first rotatable jaw 701 and the hinge bearing race 127 of the hinge flex body 107 also prevents the first rotatable jaw 701 from experiencing any translational movement during actuation of the first rotatable jaw 701. Furthermore, the aforementioned mating also defines the rotational axis 703 (FIGS. 7-11, 16A and FIG. 16C) for which the first rotatable jaw 701 and the second rotatable jaw 702 rotates about. In addition, in some embodiments, the distal end of the hinge flex body 107 contains a magnet housing aperture 190 which is configured to allow a magnet housing couplable to the first rotatable jaw 701 to enter and mate with.

In some embodiments, on the outer surface of the distal end of the hinge flex body 107 is a flex pocket 326. The flex pocket 326 is configured to allow an electrical communication component to sit within, so as to prevent any damage to said component during actuation of the first and second rotatable jaws 701, 702. In some embodiments, different electrical communication components are utilized, including but not limited to rigid flexible printed circuit boards (RFPCB), flexible printed circuit board (FPCB), and/or any other type of electrical communication component known in the art. Electrical communication components which sit in the flex pocket 326 of the hinge flex body 107 are utilized to transmit position and/or orientation data of the first rotatable jaw 701 to a central computer which processes the data and transmits control commands and/or prompts to actuators which actuate and manipulate the first rotatable jaw 701. In some embodiments, the position and/or orientation data of the first rotatable jaw 701 is obtained from magnet(s) located in a magnet housing, as well as sensors situated in the flex pocket 326 of the hinge flex body 107. In these embodiments, in addition to housing electrical communication components, the flex pocket 326 of the hinge flex body 107 contains sensors for sensing the change in magnetic field of the magnet(s) in the magnet housing as the first rotatable jaw 701 is rotated about the jaw axis 189. In some embodiments, two rotational position sensors are located in the flex pocket 326 of the hinge flex body 107 for sensing the change in magnetic field, while in other embodiments four rotational position sensors are included.

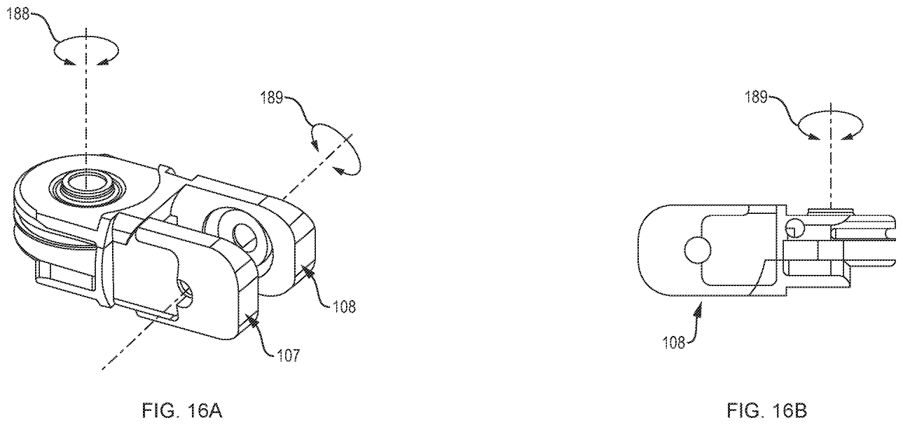

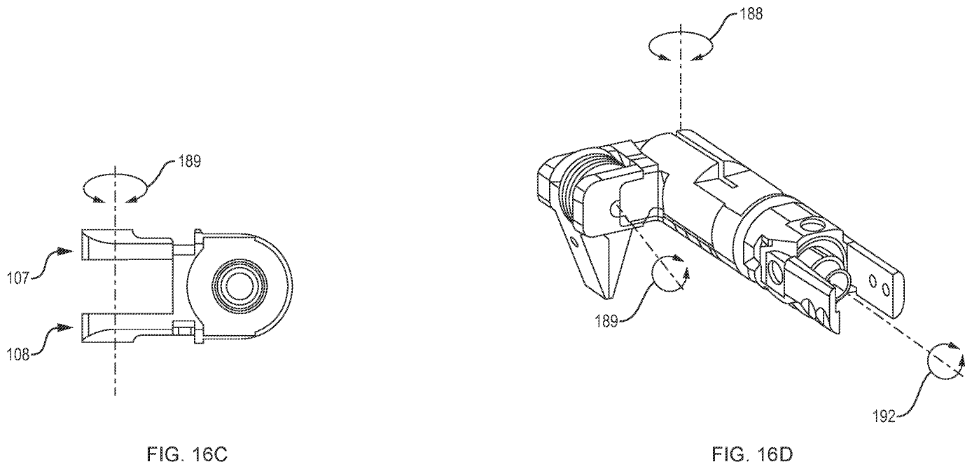

In some embodiments the hinge flex body 107 contains a proximal end. The proximal end of the hinge flex body 107 is utilized to couple and mate the end effector 700 with a hinge-rotary assembly 102, as well as defines a pitch axis 188 (FIG. 16A and FIG. 16B). As shown in FIG. 12A, in some embodiments the proximal end of the hinge flex body 107 is constructed to have two sides, an interior and an exterior side. In some embodiments, the exterior side contains a flex slot 124 which is configured to route an electrical communication component to the flex pocket 326 located on the outer surface of the distal end of the hinge flex body 107. The flex slot 124 is configured to allow an electrical communication component to sit within the slot, such that said component does not excessively bend and/or become damaged during actuation of the robotic arm 42A, 42B. In some embodiments, the flex slot 124 is located on the interior side of the proximal end of the hinge flex body 107, with the electrical communication components routed to the flex pocket 326, as detailed above. In these embodiments, the exterior side of the proximal end of the hinge flex body contains a bearing surface, thus eliminating a male bearing race 138. In addition, in these embodiments a fulcrum 328 is relocated to the interior side of the proximal end of the hinge non-flex body 108. In some embodiments, the bore 715, 715′ can mate with the fulcrum 328 as illustrated in FIGS. 7-11, 12B.

In addition, in some embodiments, the exterior side of the proximal end of the hinge flex body 107 contains a hinge hard stop 186. In these embodiments, the hinge hard stop 186 is configured to constrain the first and second rotatable jaws 701, 702 from rotating about the pitch axis 188 (FIG. 16A) past an allowable limit of articulation. In some embodiments, the allowable limit of articulation is 30 degrees, having 15 degrees of motion in either direction, while in other embodiments the allowable limit of articulation is increased and/or decreased. As seen in FIG. 12A and FIG. 12C, the hinge hard stops 186 are configured as extruded surfaces that make contact with the distal end of the hinge-rotary assembly 102 to prevent the first and second rotatable jaws 701, 702 from being actuated past an allowable degree of rotation. The proximal end of the hinge flex body 107 is configured to be circular in shape, so as to allow the hinge-rotary assembly 102 to rotate about the pitch axis 188 during actuation.

As shown in FIGS. 12A and 12D, in some embodiments the exterior side of the proximal end of the hinge flex body 107 contains connection apertures 125. The connection apertures 125 are configured to allow a male bearing race 138 from the hinge-rotary assembly 102 to mate and couple thereto. In some embodiments, the connection apertures 125 are situated on a protruded surface above the flex slot 124, such that electrical communication components can be routed through said slot without any interference from the male bearing race 138. In some embodiments, the connection apertures 125 are eliminated, with the male bearing race 138 fabricated to be a part of the proximal end of the hinge flex body 107.

In some embodiments, on the interior side of the proximal end of the hinge flex body 107 is the fulcrum 328, which protrudes from the hinge flex body 107. As depicted in the illustrative embodiment shown in FIG. 12B, the fulcrum 328 is configured to be cylindrical in shape, having an outer diameter that allows the fulcrum 328 to pass through an aperture on an idler pulley 137. In some embodiments, the fulcrum 328 is substituted for a bearing or any axle known in the art. In addition, in some embodiments the proximal end of the hinge flex body 107 contains a jaw hard stop 187. In these embodiments, the jaw hard stop 187 is configured to constrain the first rotatable jaw 701 and the second rotatable jaw 702 from rotating about the jaw axis 189 (FIG. 16A and FIG. 16C) past an allowable limit of articulation. As seen in FIG. 12B, the jaw hard stop 187 is configured as an extruded surface that makes contact with the proximal end of the first and second rotatable jaw 701, 702 to prevent the first and second rotatable jaw 701, 702 from being actuated past an allowable degree of rotation. In some embodiments, the allowable degree of rotation is about 90 degrees in either direction, while in other embodiments the allowable degree of rotation is less than or more than 90 degrees of rotation in either direction.

As mentioned above, in some embodiments the hinge flex body 107 and the hinge non-flex body 108 function as a housing for the first rotatable jaw 701 and the second rotatable jaw 702, along with other components of the end effector 700. FIGS. 13A-13D show multiple views of an illustrative embodiment of a hinge non-flex body 108. As shown in FIGS. 13A-13D, in some embodiments, the hinge non-flex body 108 is fabricated to contain a proximal and a distal end. The proximal and distal ends of the hinge non-flex body 108 are configured to have approximately the same architecture as the distal and proximal ends of the hinge flex body 107, such that the connection between the hinge flex body 107 and hinge non-flex body 108 is seamless, with the outer profiles of both bodies being flush with one another (FIGS. 16A-16C).

In some embodiments, the second rotatable jaw 702 includes a bearing race. As depicted in FIG. 13A, in some embodiments the distal end of the hinge non-flex body 108 is configured to have an inner and outer surface. In some embodiments, located on the inner surface of the distal end of the hinge non-flex body 108 is the hinge bearing race 127, which is configured to mate and couple with the bearing race of the second rotatable jaw 702. In addition to functioning as a housing for a plurality of ball bearings 109, the mating between the bearing race of the second rotatable jaw 702 and the hinge bearing race 127 of the hinge non-flex body 108 also prevents the second rotatable jaw 702 from experiencing any transitional movement during actuation of the second rotatable jaw 702. Furthermore, the aforementioned mating also defines the jaw axis 189 (FIG. 16A and FIG. 16C) for which the second rotatable jaw 702 rotates about. In addition, in some embodiments, the distal end of the hinge non-flex body 108 contains a magnet housing aperture 190 which is configured to allow the magnet housing coupled to the second rotatable jaw 702 to enter and mate with.

In some embodiments, on the outer surface of the distal end of the hinge non-flex body 108 is a flex pocket 326. The flex pocket 326 is configured to allow an electrical communication component to sit within, so as to prevent any damage to said component during actuation of the end effector 700. In various embodiment, different electrical communication components are utilized, including but not limited to rigid flexible printed circuit boards (RFPCB), flexible printed circuit board (FPCB), and/or any other type of electrical communication component known in the art. Electrical communication components situated in the flex pocket 326 of the hinge non-flex body 108 are utilized to transmit position and/or orientation data of the second rotatable jaw 702 to a central computer which processes the data and transmits control commands and/or prompts to actuators which actuate and manipulate the second rotatable jaw 702. In some embodiments, the position and/or orientation data of the second rotatable jaw 702 is obtained from magnet(s) located in magnet housing couplable to the second rotatable jaw 702, as well as sensors contained in the flex pocket 326 of the hinge non-flex body 108 and the end effector 700. In these embodiments, in addition to housing electrical communication components, the flex pocket 326 of the hinge non-flex body 108 contains sensors for sensing the change in magnetic field of the magnet(s) in magnet housing as the second rotatable jaw 702 is rotated about the axis of rotation 703. In some embodiments, two rotational position sensors are located in the flex pocket 326 of the hinge non-flex body 108 for sensing the change in magnetic field, while in other embodiments four rotational position sensors are located in the flex pocket 326. The magnet sensing is further detailed below.

As mentioned above, in some embodiments the hinge non-flex body 108 contains a proximal end. The proximal end of the hinge non-flex body 108 is utilized in conjunction with the proximal end of the hinge flex body 107 to couple and mate the end effector 700 with the hinge-rotary assembly 102, with the mating and coupling defining the pitch axis 188 (FIG. 16A and FIG. 16B). As shown in FIGS. 13A-13D, in some embodiments the proximal end of the hinge non-flex body 108 is constructed to have two sides, with one side containing a hinge bearing race 129. The hinge bearing race 129 is configured to allow the plurality of ball bearing 109 to sit in, and ride along the race during actuation of the end effector 700. The ball bearings 109 which sit in the hinge bearing race 129 of the hinge non-flex body 108 also sits within a bearing race 147 of a female hinge body 134. In some embodiments, the proximal end of the hinge non-flex body 108 contains a magnet pocket 152. In these embodiments, the magnet pocket 152 is configured allow a magnet and/or a plurality of magnets to sit in. As further detailed below, the magnet is utilized along with sensors located on the hinge-rotary assembly 102 to obtain position data and in some embodiments, orientation data of the end effector 700 as the end effector is rotated about the pitch axis 188.

In some embodiments, the outer surface of the proximal end of the hinge non-flex body 108 is fabricated to contain a cable raceway 331. The cable raceway 331 is configured to route a cable through a cable termination channel 332 situated on one side of the proximal end of the hinge non-flex body 108. The cable termination channel 332 is configured to route the cable to a cable termination site 131 located on the proximal end of the hinge non-flex body 108. In different embodiments, various methods and/or techniques, are utilized to terminate the cable in the cable termination site 131.

In some embodiments, one side of the proximal end of the hinge non-flex body 108 contains a fulcrum aperture 133 (FIG. 13C). In these embodiments, the fulcrum aperture 133 is configured to allow the fulcrum 328 of the hinge flex body 107 to enter and sit within said aperture. Furthermore, in some embodiments, the proximal end of the hinge non-flex body 108 contains a jaw hard stop 187. In these embodiments, the jaw hard stop 187 is configured to constrain the first jaw 701 and/or the second jaw 702 from rotating about the axis of rotation 703 (FIG. 16A and FIG. 16C) past an allowable limit of articulation. As seen in FIG. 13D, the jaw hard stop 187 is configured as an extruded surface that makes contact with the proximal end of the first and/or second rotatable jaw to prevent said rotatable jaws from being actuated past an allowable degree of rotation. In some embodiments, the allowable degree of rotation is 90 degrees in either direction, while in some embodiments the allowable degree of rotation is less than or more than 90 degrees of rotation in either direction.

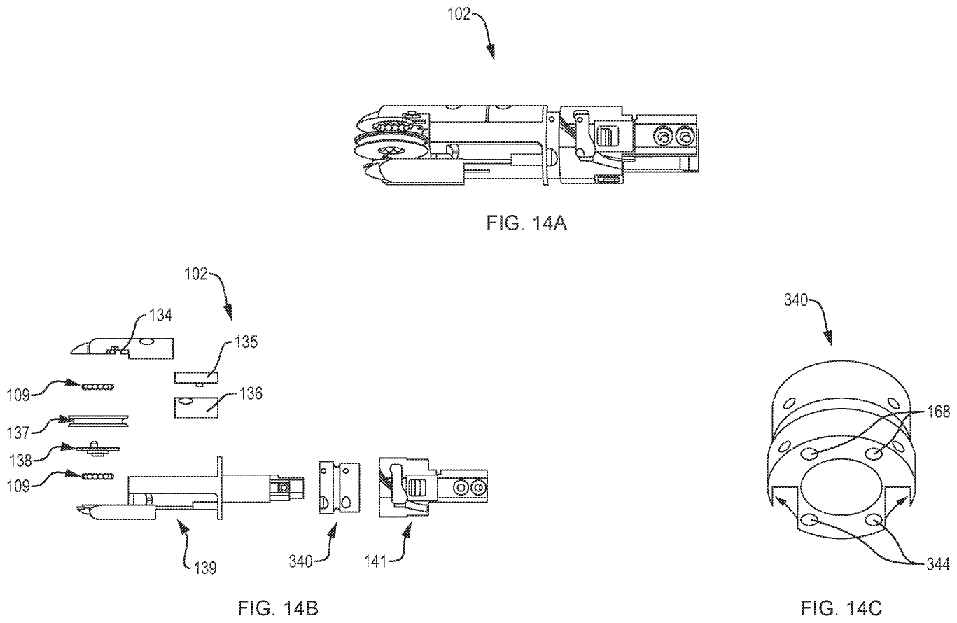

As mentioned above, the proximal ends of both the hinge flex body 107 and the hinge non-flex body 108 couple the end effector 700 to the hinge-rotary assembly 102. FIG. 14A illustrates an isometric view of an illustrative embodiment of the hinge-rotary assembly 102. FIG. 14B illustrates an exploded top view of an illustrative embodiment of the hinge-rotary assembly 102. The hinge-rotary assembly 102 is configured to provide two (2) additional degrees of freedom (DOFs), to the robotic arm 42. The hinge-rotary assembly 102 in conjunction with the hinge flex body 107 and the hinge non-flex body 108 is configured to provide one DOF, with the DOF being rotation of the end effector 700 about the pitch axis 188 (FIG. 16A). In addition, the hinge-rotary assembly 102 is also configured to provide another DOF, with this DOF being the rotation of the robotic arm 42 about a roll axis. Furthermore, in addition to providing two additional DOFs, the hinge-rotary assembly 102 also functions as an intermediary, connecting the electrical communication components of the end effector 700, with electrical communication components of the rest of the robotic arm 42, so as to allow the data obtained from sensors on the end effector 700 to be transmitted from said assembly, through the hinge-rotary assembly 102 to a central computer, as well as to allow data to be transmitted from the central computer back to the end effector 700.

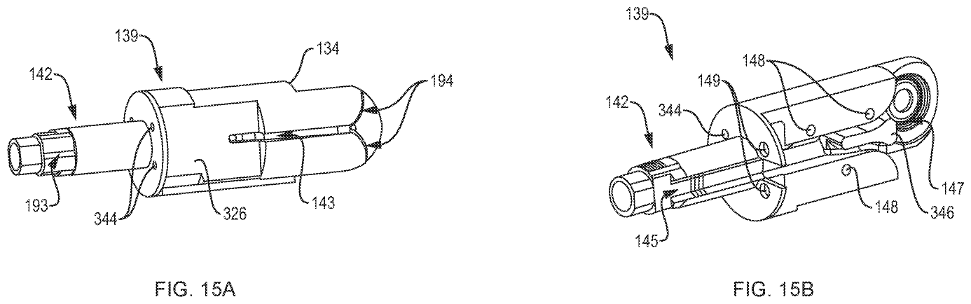

As depicted in the illustrative embodiment shown in FIG. 14B, the hinge-rotary assembly 102 contains a female hinge body, also referred to as a hinge cover 134 and a male rotary-hinge body, also referred to as a hinge-rotary body 139. The female hinge body 134 along with the male rotary-hinge body 139 are fabricated to function as a housing for various components of the hinge-rotary assembly 102, as well as a connection and mating point for the proximal ends of the hinge flex body 107 and the hinge non-flex body 108. FIG. 15A and FIG. 15B illustrate multiple views of an illustrative embodiment of the male rotary-hinge body 139. As seen in FIGS. 15A-15B, the male rotary-hinge body 139 contains a distal and proximal end, as well as outer and inner surfaces.

As depicted in FIGS. 15A-15B, the proximal end of the male rotary-hinge body, also referred to as the male hinge-rotary body 139 contains a cable conduit 142. In some embodiments, the cable conduit 142 is fabricated as a cylindrical shaft, having an aperture for one or more cables (not shown) which are routed through. The cable conduit 142 is configured to allow the aforementioned cables to be routed through the conduit, such that when the robotic arm 42A, 42B is rotated about the roll axis 192 (FIG. 16D), the cables do not become tangled with one another. In some embodiments, located on the outer surface of the cable conduit 142 is a bearing interface 193, on which a bearing (not shown) sits, the bearing is configured to allow for rotation of the cable conduit 142 through a variety of loading conditions.

As shown in FIG. 15B, in some embodiments, located on the inner surface of the cable conduit 142 is a flex guide surface 145. In some embodiments, the flex guide surface 145 is configured to be a flat surface for which an electrical communication component(s) rests on. The flex guide surface 145 is also configured to route and guide electrical communication component(s) from a robotic arm through the proximal end of the hinge-rotary assembly, where the component(s) operatively connects to other electrical communication component(s) that are routed from the end effector 700 through the distal end of the hinge-rotary assembly. The flex guide surface 145 is fabricated so electrical communication component(s) sit flat against the surface, such that during actuation of the robotic arm 42 the component(s) are not bent and/or damaged. In some embodiments, electrical communication is transmitted through the cable conduit 142 via electrical communication wires and/or cables known in the art, including but not limited to copper cables and/or fiber optic cables.

In addition, the cable conduit 142 is configured to allow a rotary pulley body 340 (FIGS. 14B, 14C) to connect and mate with the male rotary-hinge body 139. In some embodiments, the rotary pulley body 340 is fabricated to have an opening in its center to allow the cable conduit 142 to pass through. In these embodiments, the rotary pulley body 340 connects and mates with the male rotary-hinge body 139 via a screw connection, with screws passing through through-holes 149, and entering tapped holes 168 (FIG. 14C), on the rotary pulley body 340. In addition, the rotary pulley body 340 is coupled to the male rotary-hinge body 139 via a pin connection, with pins entering and sitting with pin connection apertures 344 located on both the male rotary-hinge body 139 and the rotary pulley body 340. The pin connection is configured to constrain the rotary pulley body 340 to the male rotary-hinge body 139 such that the rotary pulley body 340 does not rotate during actuation of the robotic arm 42 about a roll axis 192 (FIG. 16D). In some embodiments, the rotary pulley body 340 is fabricated as part of the male rotary-hinge body 139, thus eliminating the connections described above.

As mentioned above, the male rotary-hinge body 139 is configured to have a distal end having an inner and outer surface. As depicted in FIG. 15A, in some embodiments, located on the outer surface of the distal end of the male rotary-hinge body 139 is a cable guide slot 143. Cable guide slot 143 is configured to route and guide a cable from the hinge-rotary assembly 102 through the proximal end of the end effector 700 to the cable routing raceway 111 located on the actuation hub 180 of the first rotatable jaw 701 or the second rotatable jaw 702. In some embodiments, the cable is routed through the cable conduit 142 to the inner surface of the distal end of the male rotary-hinge body 139, where the cable passes through an aperture and enters the cable guide slot 143 on the outer surface of the distal end of the male rotary-hinge body 139. As the cable reaches the end of the cable guide slot 143, the cable is routed along decoupling surfaces 194. Decoupling surfaces 194 may be configured to decouple cable motion from joint motion, to decouple joint motion from cable motion, or to decouple cable motion from joint motion and decouple joint motion from cable motion. The decoupling surfaces are fabricated to be curved in shape, such that when the first and second rotatable jaws rotate, the cables that close the rotatable jaws 701, 702 pivot about the center of the pitch axis 188 (FIG. 16A-B, 16D) without changing length. The curve of the decoupling surfaces 194 allow the cables to wrap along it such that the cables neither lose tension nor become over tensioned, as there is no sliding motion between the cable and the surface. In addition, the decoupling surfaces effectuate decoupled motion of the rotatable jaws 701, 702 when rotated about the axis of rotation 703 and the end effector 700 when rotated about the pitch axis.

In some embodiments, also located on the outer surface of the distal end of the male rotary-hinge body 139 is the flex pocket 326. As detailed above, the flex pocket 326 is configured to allow an electrical communication component(s) to sit within and mount to the pocket, so as to prevent any damage to the component(s) during actuation of the robotic arm 42. In various embodiments, different electrical communication components are utilized, including but not limited to rigid flexible printed circuit boards (RFPCB), flexible printed circuit boards (FPCB), and/or any other type(s) of electrical communication components known in the art.

As illustrated in FIG. 15B, in some embodiments, the inner surface of the distal end of the male rotary-hinge body 139 contains a cable routing protrusion 346. The cable routing protrusion 346 is configured to direct cables routed through the distal end of the hinge-rotary assembly 102 to a desired location. In some embodiments, the cable routing protrusion 346 is fabricated to be a smooth surface so that cables are not damaged during actuation. In addition, the cable routing protrusion 346 is configured to function as a divider, so that the cables that are routed through the distal end of the male rotary-hinge body 139 do not become tangled or intertwined with each other, as well as to prevent the cables from catching or hooking on to another component of the robotic arm 42A, 42B as the cables are routed to a desired location.

In some embodiments, the cable routing protrusion 346 is configured to have two sides, with one side routing one cable to a desired location, and the other side routing a different cable to a desired location. In some embodiments, the cable routing protrusion 346 is eliminated and replace by channels and/or conduits that direct cables to a desired location.

In some embodiments, the inner surface of the distal end of the male rotary-hinge body 139 contains a plurality of tapped holes 1348. In these embodiments, the tapped holes 1348 are configured to allow a screw to enter, thus mating and coupling the female hinge body 134 with the male rotary-hinge body 139. In addition, in some embodiments, one or more of the plurality of tapped holes 1348 are utilized to mate and couple a bottom flex cover 136 (FIG. 14B) to the male rotary-hinge body 139.

In some embodiments, the inner surface of the distal end of the male rotary-hinge body 139 includes a bearing race 147 (FIG. 15B). The bearing race 147 is configured to allow a plurality of ball bearings 109 (FIG. 14B) to sit within, and ride along during articulation of a robotic arm 42A, 42B. In these embodiments, the ball bearings 109 that sit within the bearing race 147 of the male rotary-hinge body 139, also sit on a bearing raceway located on a male bearing race 138. In these embodiments, the male bearing race 138 is configured to couple to the bearing race 147 of the distal end of the male rotary-hinge body 139, as well as with the proximal end of the hinge flex body 107. Further disclosure regarding control of movement of end effectors of a robotic assembly, including a magnetic housing and bearing race, is provided in U.S. Pat. No. 11,576,732 B2, which is incorporated by reference herein in its entirety.

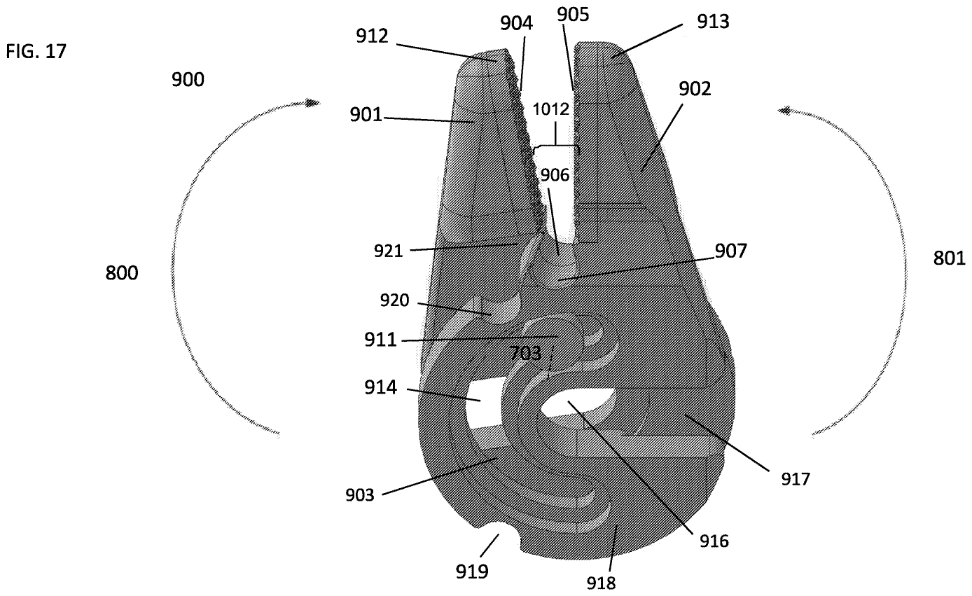

FIG. 17-23 illustrates another embodiment of a surgical end effector 900 as taught herein. FIG. 17 illustrates an end effector 900 as taught herein. The end effector 900 includes a first rotatable jaw 901 opposed to a second rotatable jaw 902 that are pivotally coupled by a fastener 911 that extends through the first and second rotatable jaws 901, 902. Both rotatable jaws rotate about the fastener 911, which defines to an axis of rotation 703 and are adapted to clamp tissue and other material therebetween or cut tissue or material depending on how the first and second rotatable jaws 901, 902 are rotated relative to each other. In some embodiments, one rotatable jaw has a bore through which the fastener extends and one rotatable jaw has a radially shaped channel (race) 914 through which the fastener extends. The rotatable jaw with the radially shaped channel 914 rotates about the fastener via the channel. The radially shaped channel 914 provides a rotational guide for the rotatable jaw. The first rotatable jaw 901 includes a first grasping face 904, a first cutting face 906, a first cutting edge 908 and a distal tip 912. The second rotatable jaw 902 includes a second grasping face 905, a second cutting face 907, a second cutting edge 909, and a distal tip 913. The first and second rotatable jaws 901, 902 each include a slot 916, 917, respectively. The slots 916, 917 are mateable with a raised or protruding boss element 330 located at a distal end of one of the robotic arms 42A, 42B to control rotation of the first and second rotatable jaws 901, 902 either individually or in unison. The boss element 330 is described in more detail below. The boss element 330 is coupled to a pulley element 320.

In some embodiments, the end effector 900 is removably attached to the surgical robotic system 10, described above, via a cartridge. The cartridge is used for securing the end effector 900 and releasing the end effector 900 when engaged by a distal end of a robotic arm.

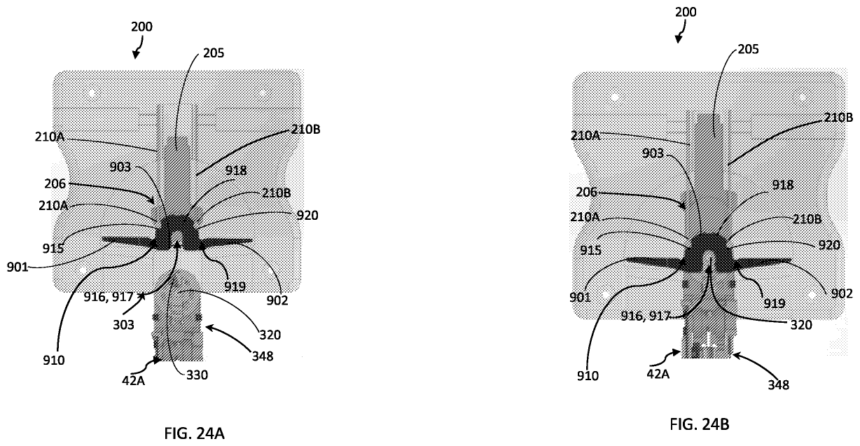

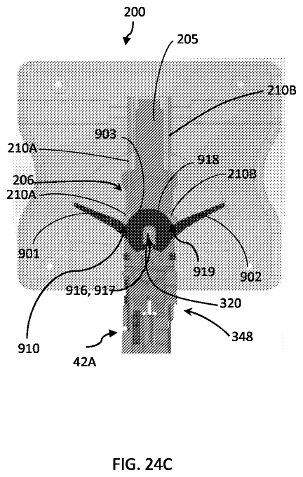

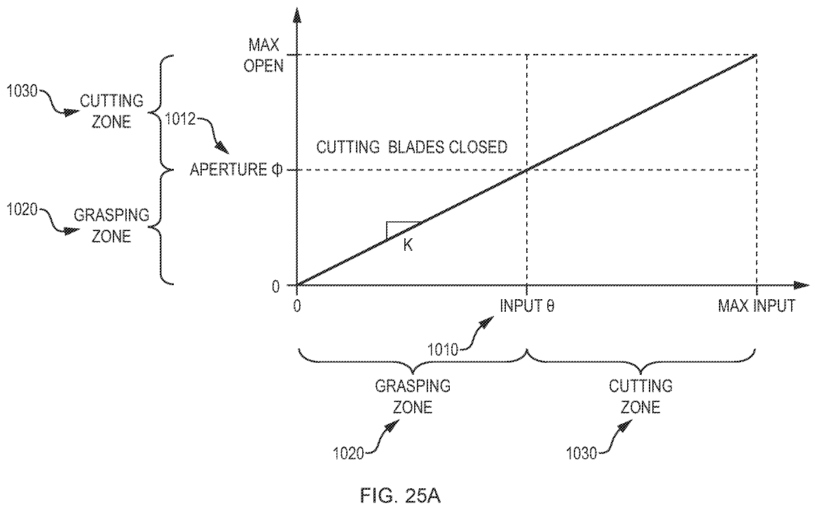

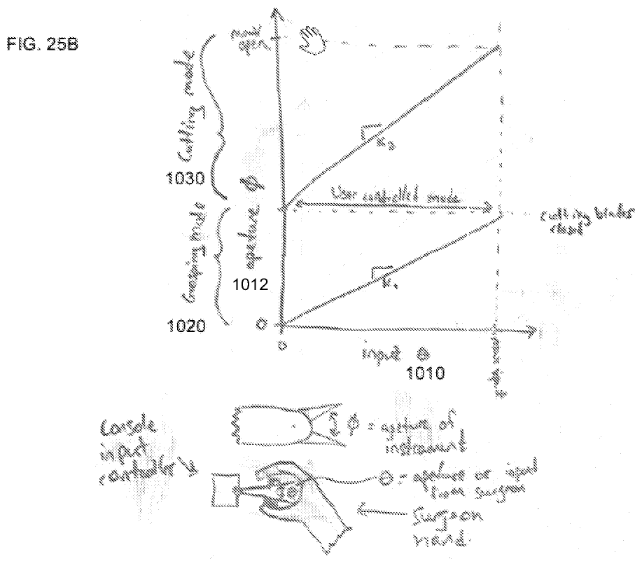

The first rotatable jaw 901 includes a main body 903. The main body 903 includes a first notch 910 and a second notch 915 to accommodate detents for a tool exchange via the cartridge. The second rotatable jaw 902 includes a main body 918. The main body 918 includes a first notch 919 and a second notch 920 to accommodate detents for the tool exchange via the cartridge. Notches 910, 915, 919, 920 engage with detents discussed below in relation to FIGS. 24A-C (not shown), securing the end effector 900 on the cartridge. The cooperative engagement and disengagement of the end effector 900 to a distal end of one of the robotic arms 42A via the cartridge is described below in relation of FIGS. 24A-C. The cooperative engagement and disengagement of the notches 910, 915, 919, 920 on the first and second rotatable jaws 901, 902 to corresponding detents in the cartridge allow the end effector 900 to be securely held in the cartridge when not in use and to be removed from the cartridge when needed. In some embodiments, the detents have a shape of any of a ball, a latch, a hook, or a protrusion and the notches 910, 915, 919, 920 have a corresponding same to receive the detents. The notches 910, 915, 919, 920 are placed behind the first and second cutting faces 906, 907 to accommodate the cartridge.

The cutting faces 906, 907 are positioned close to the base of the first and second rotatable jaws 901, 902 respectively such that they are on the rights side of the detent. This prevents the cutting faces 906, 907 from interfering with the cartridge.

In some embodiments either the first rotatable jaw 901, the second rotatable jaw 902, or both, include a textured surface on the grasping face 904, 905. The first and second grasping faces 904, 905 form a first pair of opposing features of the end effector 900. The first cutting face 906 and the first cutting edge 908 together with the second cutting face 907 and the second cutting edge 909 form a second pair of opposing features of the end effector 900.

The first cutting face 906 is positioned below the second grasping face 905, is substantially parallel to the second cutting face 907, and is located distally from the tip portion 912. Likewise, the second cutting face 907 is positioned below the first grasping face 904, is substantially parallel to the first cutting face 906, and is located distally from the tip portion 913. The positioning of the cutting faces 906, 907 relative the grasping face on the opposite jaw allows rotational movement of the first and second rotational jaws 901, 902 without interference from the second pair of opposing features. For example, the first cutting face 906 rotates parallel to the second rotatable jaw 902 which allows the first cutting face 906 to rotate without interfering with the second rotatable jaw 902. Further, having the first and second cutting faces 906, 907 and, in turn, the first and second cutting edges 908, 909 set back from the tip portion 912, 913 of each of the first grasping face 904 and the second grasping face 905 keeps the cutting features of the end effector 900 out of the way of a grasping operation.

The first grasping face 904 includes a notched or cutout portion 921 located distally from the tip portion 912. Likewise, the second grasping face 905 includes a notched or cutout portion 922 located distally from the tip portion 913. The notched portions 921, 922 of the first and second grasping faces 904, 905 allow rotational movement of the cutting face 906, 907 on the opposing jaw. For example, the first grasping face 904 includes the notch 921 which allows the second cutting face 907 to rotate without interfering with the first rotatable jaw 901. Further, having the first and second cutting faces 906, 907 and, in turn, the first and second cutting edges 908, 909 set back from the tip portion 912, 913 of each of the first grasping face 904 and the second grasping face 905 keeps the cutting features of the end effector 900 out of the way of a grasping operation. The setback location of the first and second cutting faces 906, 907 and the first and second cutting edges 908, 909 help avoid accidental or unintentional cutting during a grasping operation.

In some embodiments, the cutting faces 906, 907 are contoured, for example, they have a curved or helical shape to facilitate holding of a suture during a cutting operation. In some embodiments, the cutting faces 906, 907 are substantially flat.

In FIG. 17, the grasping operation is illustrated. The first rotatable jaw 901 is rotating in the first rotational direction 800, for example, a clockwise direction. The second rotatable jaw 902 is rotating in the second rotational direction 801, for example, a counterclockwise direction. In other words, the grasping face 904 of the first rotatable jaw 901 and the grasping face 905 of the second rotatable jaw 902 are rotating toward each other in a closing direction to grasp or clamp an object between the first grasping face 904 and the second grasping face 905. In some embodiments, the grasping face 904 of first rotatable jaw 901 is able to rotate toward the grasping face 905 of the second rotatable jaw 902 while the second rotatable jaw 902 is stationary in order to grasp an object. Likewise, in some embodiments, the grasping face 905 of the second rotatable jaw 902 is able to rotate toward the grasping face 904 of the first rotatable jaw 901 while the first rotatable jaw 901 is stationary in order to grasp an object.

During the grasping operation, the cutting faces 906, 907 do not pass by each other. The first cutting face 906 is disposed below a portion of the first grasping face 904 and the second grasping face 905. The second cutting face 907 is disposed below a portion of the first grasping face 904 and the second grasping face 905. During the grasping operation, the second cutting face 907 rotates in the second rotational direction 801 while the first cutting face 906 rotates in the first rotational direction 800, thereby rotating away from each other.

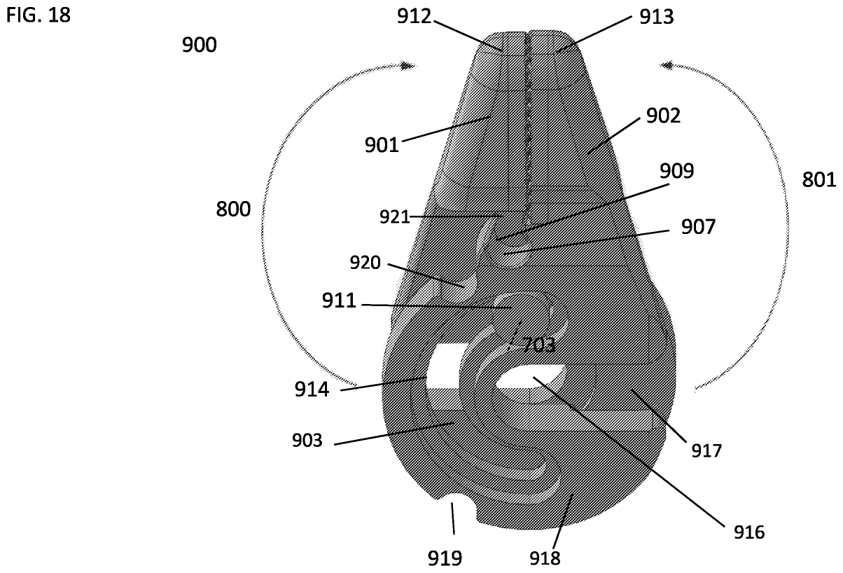

FIG. 18 illustrates the end effector 900 in a fully closed state. In the fully closed state, the cutting faces 906, 907 are located on opposite sides of the first and the second grasping faces 904, 905. As such, the cutting faces 906, 907 are spaced apart and at least partially shielded by the notched or cutout portions 921, 922 by the opposing jaw during the grasping operation. The first and the second grasping faces 904, 905 include the notched areas 921, 922 allowing first and the second grasping faces 904, 905 to rotate past the cutting faces 906, 907 during the grasping operation. The first and second rotatable jaws 901, 902 are able to rotate in a manner to perform a grasping operation and simultaneously rotate the cutting faces 906, 907 out of the way during the grasping operation. The first and the second rotatable jaws 901, 902 close toward each other to receive tissue and other material therebetween.

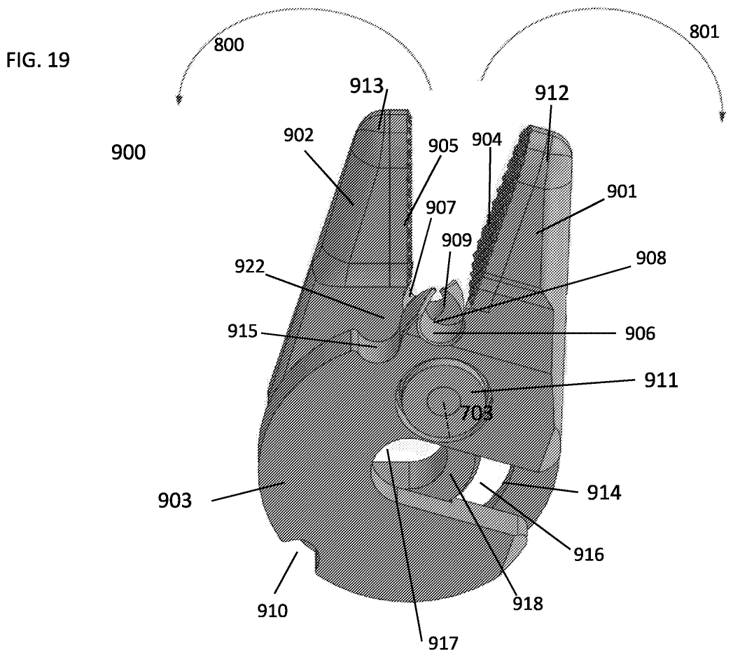

FIGS. 19-22 illustrate the first rotatable jaw 901 rotating in the second rotational direction 801 and the second rotatable jaw 902 rotating in the first rotational direction 800 to perform the cutting operation of the end effector 900. As in the grasping operation, the first and the second rotatable jaws 901, 902 rotate relative to each other. As mentioned above, the first rotatable jaw 901 and the second rotatable jaw 902 are able to rotate independently of each other or in unison. In some embodiments, during the cutting operation, the first cutting face 906 rotates in the second rotational direction 801 and the second cutting face 907 rotates in the first rotational direction 800. In some embodiments, during the cutting operation, the first cutting face 906 rotates in the second rotational direction 801 and the second cutting face 907 does not rotate. In some embodiments, during the cutting operation, the first cutting face 906 does not rotate and the second cutting face 907 rotates in the first rotational direction 800. Whereas during the grasping operation the cutting faces 906, 907 are rotated away from each other, while the first and second grasping faces 904, 905 are rotated away from each other. During the cutting operation the cutting faces 906, 907 are rotated toward each other and eventually past each other in order to cut. In some embodiments, the first and second rotatable jaws 901, 902 are able to open about 30 degrees relative to each other. This is advantageous because the surgical end effector has a greater range of motion without potentially making contact with tissue within the cavity while performing the intended operation. With a smaller opening range, the cutting faces 906, 907 are exposed less. As the first and the second rotatable jaws 901, 902 rotate in the second and the first rotational direction 801, 800 respectively, the cutting faces 906, 907 rotate past each other to cut, for example, a suture. As the cutting faces 906, 907 rotate past each other, the object resting therein (such as a suture) is cut by the cutting edge 908, 909.

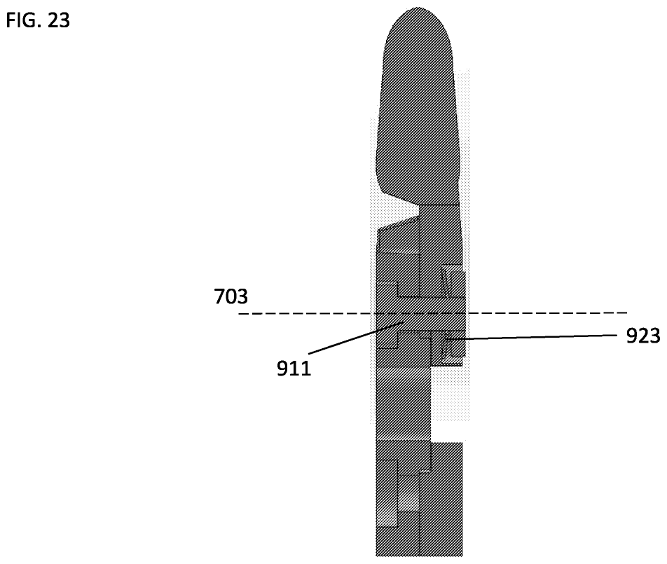

FIG. 23 illustrates an end view of the surgical end effector 900. The surgical end effector 900 includes a frusto-conical shaped washer 923 to apply a preload force to execute the cutting and grasping operation. The frusto-conical shaped washer 923 is coaxial with the fastener 911 and held in place by the fastener 911. The frusto-conical shaped washer 923 is able to apply a high force with a short spring length and minimal movement when compressed. This is advantageous because only a small range of motion is necessary to open or close the rotatable jaws.

With respect to the end effector described in FIGS. 17-23, below is a description of an embodiment of a suitable attachment mechanism. The attachment mechanism allows the end effector 900 to connect and disconnect from a distal end of a robotic arm. Some systems and methods taught herein employ cartridges that hold the end effector with corresponding mating features and facilitate installing and removing the end effector on a distal end of the robotic arm to form a functional end effector tool. In some embodiments, the cartridge is designed to securely hold the end effector to permit easy retrieval of the end effector and installation on a distal end of a robotic arm forming a functional tool for use in a surgical procedure. Because the end effector is held within the cartridge, the end effector may be retained in a sanitary and/or sterile condition and may be protected from damage due to being dropped, impacted by other objects, or other contact.

In some embodiments, the cartridge may include a cartridge body with an opening therein for insertion of a distal end of the robotic arm. The cartridge may also include a holder which includes a holder channel and cradle portion. The cradle portion is configured to hold a surgical tool. For example, the cradle portion engages with at least a portion of the end effector. In some embodiments, the cradle portion includes an upper cradle portion and a lower cradle portion for engaging and holding the end effector. The holder also includes a pair of spring tabs. Each spring tab includes a detent disposed at a proximal end of the spring tab and each spring tab is secured to the cartridge body at a distal end. Each detent is configured to engage a corresponding notch in the end effector body of the corresponding end effector. The cartridge may also include a spring connected to the holder and the cartridge body, permitting movement of the holder relative to the cartridge body upon compression and extension of the spring. In some embodiments, the holder also includes a pair of spring tab channels, each of the pair of spring tabs extending through a corresponding one of the spring tab channels, where the spring tab channels are configured to enable the holder to move along an axis a central axis of a holder channel of the cartridge body with respect to the pair of spring tabs while restricting lateral deflection of a portion of the spring tabs positioned within the spring tab channels. Insertion of a robotic arm and the force of the robotic arm against the holder causes the holder to move along the central axis, permitting the detents of the spring tabs to deflect and disengage from the notches of the end effector, thereby releasing the end effector after they are engaged with the robotic arms. A detent is configured to engage with a corresponding notch on the end effector mechanism. Detents are sized and positioned to engage with notches of the end effector mechanism when the mechanism is held within the arm. In some embodiments, the detents have a circular or semicircular profile.

FIGS. 24A to 24C illustrate the process by which a distal end of one of the robotic arms 42A, 42B can engage with the first and second rotatable jaws 901, 902 held within the cartridge 200 in accordance with some embodiments taught herein. For ease of discussion with respect to FIGS. 24A to 24C a single robotic arm 42A is discussed. This is not meant to be limiting and the below discussion is equally applicable to a second or third or fourth robotic arm. Some embodiments disclosed herein employ cartridges that hold an end effector disclosed herein with corresponding mating features and facilitate installing the end effector on a distal end of a robotic arm to form a functional end effector tool. The cartridges also function to receive and hold an end effector from the distal end of the robotic arm enabling different end effectors held in a different cartridge to be installed on the same robotic arm. In some embodiments, the cartridge is designed to securely hold an end effector to permit easy retrieval of the end effector and installation on a distal end of a robotic arm forming a functional tool for use in a surgical procedure. The end effector may also be configured to include conductive contact elements. The cartridges suitable for use with an end effector as taught herein may include internal structures designed to hold an end effector within a tool cavity of the cartridge to permit retrieval of the end effector by a robotic arm. FIGS. 17-23 illustrates an example end effector configured to be held by a cartridge in accordance with some embodiments. The first and second rotatable jaw 901, 902 include the main body 903, 918 that defines the slot 916, 917 and includes the notches 910, 915 and 919, 920 respectively. The embodiments of the end effector 900 illustrated in FIGS. 17-23 are able to attach and detach to the end of the robotic arms 42A, 42B as discussed below.

In FIG. 24A, the distal end 348 of the robotic arm 42A is inserted into the cartridge 200 via an opening in a side surface. The opening in the side surface may be outfitted with a reusable door or a single use covering material, such as a plastic sheet to provide protection from dust or other contaminates while still permitting entry of the distal end 348 of the robotic arm 42A as illustrated in FIG. 24A. FIG. 24A also shows the distal end 348 of the robotic arm 42A featuring pulley elements 320 and raised or protruding boss element 330.

FIG. 24B illustrates the further insertion of the distal end 348 of the robotic arm 42A such that a boss element 330 of the distal end 348 of the robotic arm 42A fits within the slot 916, 917 of first and second rotatable jaws 901, 902. As illustrated in FIG. 24C, the distal end 348 of one of the robotic arm 42A is inserted further into the cartridge 200 pushing against a holder 205 to compress a spring and permit the holder 205 to move away from the opening. Holder 205 includes spring tab channels 206 through which the spring tabs 210A, 210B extend and limits deflection of the spring tabs 210A, 210B. Each spring tab includes a detent 312A, 312B at a first end of the spring tab, which may be referred to as a proximal end of the spring tab. Each detent is configured to engage a corresponding one of the notches 910, 915, 919, 920 on the end effector 900. Each spring tab 210A, 210B is connected to the cartridge body at a second end 211A, 211B, which may be described as a distal end in some embodiments. The spring tab channels 206 are configured to enable the holder 205 to move along a central axis of the holder channel 209 with respect to the pair of spring tabs 210A, 210B while restricting lateral deflection of a portion of the spring tabs positioned within the spring tab channels 206. As the holder 205 recedes into the holder channel, the end portions of spring tabs 210A, 210B featuring detents 312A, 312B are released from the spring tab channels 206 of the holder 205 and are thus able to flex.

As shown in FIG. 24C, a portion of each of the spring tabs 210A, 210B including the detents 312A, 312B flexes outwardly due to the force from the body of the first and second rotatable jaws 901, 902 when they are no longer held in position by the holder 205. As the detents 312A, 312B on the spring tabs 210A, 210B deflect outward, they disengage from corresponding notches 910, 915, 919, 920 of the first and second rotatable jaw 901, 902 thereby permitting the end effector 900 to be withdrawn from cradle portion 207 of the holder 205. After the distal end 348 of one of the robotic arms 42A, 42B has engaged the end effector 900 and it has been released from the corresponding detents 312A, 312B, the end effector 900 may be withdrawn from the cartridge body via the opening 303 of the cartridge body. In some embodiments, the robotic arm 42A may rotate the end effector 900 to close the arms and reduce the width of the end effector 900 to permit easier withdrawal through opening 303.

Some embodiments include inserting the distal end of a robotic arm into the cartridge, including engaging the distal end of the robotic arm with an end effector. For example, a boss on the distal end may engage with a slot of the tool. The engagement may occur via friction between surfaces of the two components. Some embodiments include further inserting the distal end of the arm to displace the holder and compress a spring. As the holder moves in this direction, the holder moves away from the details located on the end of the spring tabs, and permits movement of the spring tabs. Some embodiments include actuating the end effectors to flex detents of the spring tabs away from corresponding notches in the end effector. Some embodiments include withdrawing the arm from the cartridge and thus removing the end effector.

It is also contemplated in the present disclosure that the end effector may be replaced within the cartridge. Replacing the end effector within the cartridge may be performed, for example, during a procedure, when it is desired to switch the end effector being used on the mechanical arm and to maintain the end effector within a sterile, protected environment while the second end effector is in use, such that, optionally, the first end effector may be reattached to the arm and a subsequent phase of the procedure for further use of the end effector. Additionally, replacing the end effector within the cartridge may permit easier handling of the end effector, for example by maintaining an end effector and any bodily fluids or other bodily matter they are on within the cartridge to facilitate cleaning of the surgical robot after the procedure has been completed, or during the procedure as necessary.

The cartridge body and cartridge supports may be made of a plastic material, such as such as PET (polyethylene terephthalate), HDPE (high-density polyethylene), PVC (polyvinyl chloride), PP (polypropylene), or PS (polystyrene). Alternatively, the cartridge body and cartridge supports may be made of metals including steel, stainless steel, aluminum, nickel, copper, zinc, tin and alloys (including brass, nickel-chromium alloys, etc.).

The spring tabs may be made from any material or materials with sufficient mechanical properties to hold the end effector elements in place by engaging the detents of the spring tabs with the corresponding notches of the end effector elements. In some embodiments, the spring tabs may be made of spring steel ranging from 0.02 to 0.03 inches thick by 3 mm height. Alternatively, they may be made of other metals including stainless steel, aluminum, nickel, copper, zinc, tin and alloys (including brass, nickel-chromium alloys, etc.). A variety of types of springs may be used, such as compression springs, expansion springs (e.g., mounted on one side to the side of the cartridge containing the opening), torsion springs (e.g., torsion springs housed within the supports), etc. Alternatively, the springs and spring tabs may be made of a flexible plastic material, such as a natural or synthetic rubber, PET (polyethylene terephthalate), HDPE (high-density polyethylene), PVC (polyvinyl chloride), PP (polypropylene), or PS (polystyrene).

What is claimed is:

1. A surgical end effector, comprising:
a first jaw rotatable in a first rotational direction about an axis of rotation to perform a grasping operation and rotatable in a second rotational direction about the axis of rotation to perform a cutting operation; and
a second jaw rotatable in the second rotational direction about the axis of rotation to perform the grasping operation and rotatable in the first rotational direction about the axis of rotation to perform the cutting operation.

2. The surgical end effector of claim 1, further comprising a main body.

3. The surgical end effector of claim 1, wherein the first rotational direction comprises movement in a clockwise direction.

4. The surgical end effector of claim 1, wherein the second rotational direction comprises movement in a counterclockwise direction.

5. The surgical end effector of claim 1, wherein the first jaw and the second jaw each have a respective grasping face for contacting an object.

6. The surgical end effector of claim 4, wherein the grasping face of the first jaw or the grasping face of second rotatable jaw is a textured contact surface.

7. The surgical end effector of claim 1, wherein the first and second rotatable jaw each include a cutting face.

8. The surgical end effector of claim 7, wherein the cutting face is positioned toward the main body.

9. The surgical end effector of claim 7, wherein the cutting face of the first rotatable jaw or the second rotatable jaw is substantially flat.

10. The surgical end effector of claim 7, wherein the cutting face of the first rotatable jaw or the second rotatable jaw is contoured.

11. The surgical end effector of claim 1, wherein the first and second rotatable jaw are grasper jaws.

12. The surgical end effector of claim 1, wherein the first and second rotatable jaw are needle driver jaws.

13. The surgical end effector of claim 7, wherein the first and second rotatable jaw further comprise a notched area for the cutting face to rotate through.

14. The surgical end effector of claim 1, wherein the first rotatable jaw includes a cutting edge and the second rotatable jaw includes a cutting edge for cutting.

15. The surgical end effector of claim 14, wherein the cutting edge of the first rotatable jaw and the cutting edge of the second rotatable jaw are positioned relative to each other to cut when the first and second rotatable jaw are performing the cutting operation.

16. The surgical end effector of claim 15, wherein the first rotatable jaw and the second rotatable jaw each have a respective grasping face for contacting an object and the cutting edge of the first rotatable jaw and the cutting edge of the second rotatable jaw are positioned relative to each other such that each of the cutting edges are distally disposed from the grasping faces of the first and second rotatable jaws.

17. The surgical end effector of claim 1, wherein the first rotatable jaw and the second rotatable jaw rotate about the axis of rotation independently of each other.

18. The surgical end effector of claim 1, wherein the first rotatable jaw rotates about the axis of rotation in unison with the second rotatable jaw.

19. The surgical end effector of claim 16, wherein a location of the cutting edge of the first rotatable jaw and the cutting edge of the second rotatable jaw relative to a location of the grasping faces of the first and second rotatable jaws allows the cutting edge of the first rotatable jaw and the cutting edge of the second rotatable jaw to avoid interference with an object being grasped.
